(19) United States
(12) Patent Application Publication
(10) Pub. No.: US 2021/0115423 A1
(43) Pub. Date: Apr. 22, 2021

(54) BACTERIAL MANNANASES

(71) Applicant: AB Enzymes Oy, Rajamäki (FI)

(72) Inventors: Taija Leinonen, Rajamäki (FI); Leena Valtakari, Rajamäki (FI); Michael Seefried, Darmstadt (DE); Kari Juntunen, Rajamäki (FI); Kristiina Järvinen, Espoo (FI); Daniela Dollak, Darmstadt (DE); Patrick Lorenz, Lorsch (DE); Jari Vehmaanperä, Rajamäki (FI); Pentti Ojapalo, Rajamäki (FI); Terhi Puranen, Rajamäki (FI); Daniela Herbst, Düsseldorf (DE); Susanne Wieland, Düsseldorf (DE); Nina Mussmann, Düsseldorf (DE)

(21) Appl. No.: 16/500,224

(22) PCT Filed: Apr. 5, 2017
(86) PCT No.: PCT/FI2018/050229

§ 371 Date: Oct. 2, 2019

(30) Foreign Application Priority Data
Apr. 5, 2017 (EP) 17164880.1

(51) Int. Cl. C12N 9/24; C11D 3/386; C09K 8/035; A23K 20/189; A23K 20/147; A23K 50/75; A23F 5/24; A23L 2/04; A23L 2/84; A23C 11/10; A23L 11/30

(52) U.S. Cl. CPC C12N 9/2494; C12Y 302/01078; C11D 3/38636; C09K 8/035; A23K 20/189; A23K 20/147; A23V 2002/00; A23F 5/246; A23L 2/04; A23L 2/84; A23C 11/103; A23L 11/33; A23K 50/75

(57) ABSTRACT

The present description is related to novel mannanases, compositions including mannanase, to methods for producing mannanases and to methods of using mannanases to degrade and modify mannan containing material.

### FIELD

The aspects of the disclosed embodiments relate to bacterial mannanase enzymes. The mannanases are useful in industrial applications wherein degradation or modification of mannan is desired, such as in laundry and cleaning applications, in feed, food, pulp and oil industry. The aspects of the disclosed embodiments also provide useful mannanases enzymes, polynucleotides encoding these enzymes, enzyme compositions and methods for their production and use.

### BACKGROUND

Mannans are mannose containing polysaccharides found in various plants. Mannans are poorly soluble in an aqueous environment and their physicochemical properties give rise to viscous dispersions. Additionally, mannans have high water binding capacity. All of these characteristics cause problems in several industries including brewing, baking, animal nutrition, and laundry and cleaning applications.

In plant-based diets different β-mannans are present and depending on their amounts and properties they can compromise nutrient digestion, microbial colonisation and growth performance. Enzymatic degradation of mannans reduces digesta viscosity of high water soluble mannans and leads to production of manno-oligosaccharides that may form water-insoluble linear mannans present in leguminoseae. Mannanase increases average daily gain, feed efficiency, weight uniformity and livability in all monogastric animals.

For animal feed applications, such as feed for monogastric animals with cereal diets, mannan is a contributing factor to viscosity of gut contents and it thereby adversely affects the feed digestibility and animal growth rate. For ruminants, mannan represents a substantial component of fiber intake and a more complete digestion of mannan would facilitate higher feed conversion efficiencies.

For laundry and cleaning applications enzyme compositions comprising mannanase can be used to degrade mannan. However, providing mannanases that are stable in varying storage and use conditions while still showing good mannan degrading activity is difficult.

It is an object of the aspects of the disclosed embodiments to provide novel enzymes exhibiting mannanase activity when applied in different industrial processes, as well as enzyme compositions for mannan degradation or modification.

### SUMMARY

According to the first aspect of the disclosed embodiments there is provided an enzyme composition comprising at least one mannanase enzyme having an amino acid sequence which has at least 70% sequence identity with SEQ ID NO: 16 (Man7), at least 93% sequence identity with SEQ ID NO: 12 (Man6), and/or at least 79% sequence identity with SEQ ID NO: 20 (Man14).

According to another aspect of the disclosed embodiments there is provided an enzyme composition comprising at least one mannanase enzyme with a core region having an amino acid sequence which has

at least 79% sequence identity with the amino acids 27-331 of Man7 SEQ ID NO: 16;
at least 95% sequence identity with the amino acids 35-324 of Man6 SEQ ID NO: 12; and/or
at least 85% sequence identity with the amino acids 17-314 of Man14 SEQ ID NO: 20.

In an embodiment the at least one mannanase enzyme has a core region as defined above.

The present enzyme composition is advantageous in having good stability and mannanase activity in detergents and in formulations. It is also suitable for various industrial applications wherein mannan degradation or modification is desired. The mannanases of the enzyme composition of the aspects of the disclosed embodiments are suitable for degrading and modifying mannan containing material in various chemical environments.

As evidenced by the Examples, the mannanases comprised in the enzyme composition according to the aspects of the disclosed embodiments have a structure and properties that allow production in recombinant host cells and make them useful in enzyme compositions for industrial applications. A common structural element shared by Man6, Man7 and Man14 is the GH5 domain. Another common structural element is a sequence identity of 60% between Man6 and Man7, a sequence identity of 57% between Man6 and Man14 and sequence identity of 69% between Man7 and Man14. Another common structural characteristic is the core region. These structural elements are characteristic for the mannanases of the aspects of the disclosed embodiments.

According to the second aspect there is provided a recombinant host cell comprising genetic elements that allow producing at least one recombinant polypeptide having mannanase activity and

at least 70% sequence identity with the amino acid sequence of SEQ ID NO: 16,
at least 93% sequence identity with the amino acid sequence of SEQ ID NO: 12, and/or
at least 79% sequence identity with the amino acid sequence of SEQ ID NO: 20, and wherein the host cell is selected from the group consisting of:
fungal cells,
filamentous fungal cells from Division Ascomycota, Subdivision Pezizomycotina; preferably from the group consisting of members of the Class Sordariomycetes, Subclass Hypocreomycetidae, Orders Hypocreales and Microascales and Aspergillus, Chrysosporium, Myceliophthora and Humicola;
more preferably from the group consisting of Families Hypocreacea, Nectriaceae, Clavicipitaceae, Microascaceae, and Genera Trichoderma (anamorph of Hypocrea), Fusarium, Gibberella, Nectria, Stachybotrys, Claviceps, Metarhizium, Villosiclava, Ophiocordyceps, Cephalosporium, and Scedosporium;
more preferably from the group consisting of Trichoderma reesei (Hypocrea jecorina), T. citrinoviridae, T. longibrachiatum, T. virens, T. harzianum, T. asperellum, T. atroviridae, T. parareesei, Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Acremonium (Cephalosporium) chrysogenum, and Scedosporium apiospermum, and Aspergillus niger, Aspergillus awamori, Aspergillus oryzae, Chrysosporium lucknowense, Myceliophthora thermophila, Humicola insolens, and Humicola grisea,
bacterial cells, preferably gram positive Bacilli such as B. subtilis, B. licheniformis, B. megaterium, B. amyloliquefaciens, B. pumilus, gram negative bacteria such as Escherichia coli, actinomycetales such as Streptomyces sp., and
yeasts, such as Saccharomyces cerevisiae, Pichia pastoris, Yarrowia lipolytica, most preferably Trichoderma reesei or Bacillus.

The recombinant host cell can be used to produce mannanase and to carry the polynucleotide encoding mannanase. The recombinant host cell is useful also in preparation of mannanases with different properties. For example, a host cell can be selected, which provides post-translational modifications beneficial for stability or activity, or which facilitates post-processing and formulation of mannanase produced in the host cell.

According to the third aspect is provided a recombinant polypeptide having mannanase activity and obtainable by using the host cell of the second aspect.

The recombinant polypeptide may have structural or functional properties that differentiate it from a native polypeptide having the same or similar amino acid sequence. For example, a host cell can be selected which provides the produced recombinant polypeptide with post-translational modifications, a lack thereof, or localization to facilitate production and/or formulation of the recombinant polypeptide.

According to the fourth aspect is provided a method for producing mannanase comprising:

a. cultivating a recombinant host cell of the second aspect, wherein
i. the genetic elements comprise at least one control sequence which controls the production of the recombinant polypeptide in the recombinant host cell under conditions that allow production of the polypeptide;
ii. the genetic elements optionally comprise at least one sequence encoding a signal sequence for transporting the polypeptide outside the host cell; and
iii. cultivating is carried out in conditions allowing production of the polypeptide; and
b. recovering the polypeptide.

The method provides an efficient way to produce mannanase. Because the mannanase is produced in a recombinant host cell, a mannanase production system is provided which can be optimized, tailored, and controlled in a desired manner. The mannanase produced by the method may differ from natural mannanases at a structural level. The mannanase produced by the method can e.g. have a glycosylation pattern, or other post translational modification, which causes differences in the structure and/or function when compared to a natural mannanase, such as a mannanase having similar or the same amino acid sequence, or compared to a mannanase having the same amino acid sequence but produced in another host cell. The mannanase produced by the method can be used as such or formulated into a selected formulation.

According to another aspect is provided an enzyme preparation comprising a recombinant polypeptide having mannanase activity and obtainable by using the host cell of the second aspect.

The enzyme preparation or composition may further comprise other enzyme(s) selected from the group consisting of proteases, amylases, cellulases, lipases, xylanases, mannanases, cutinases, esterases, phytases, DNAses, pectinases, pectinolytic enzymes, xanthanases, xyloglucanases, laccases, peroxidases and oxidases with or without a mediator, as well as suitable additives selected from the group consisting of stabilizers, buffers, surfactants, bleaching agents, mediators, anti-corrosion agents, builders, anti-redeposition agents, optical brighteners, dyes, pigments, perfumes, caustics, abrasives and preservatives.

According to a fifth aspect is provided a method for degrading or modifying mannan containing material comprising treating said ss mannan containing material with an effective amount of the present enzyme composition or the recombinant polypeptide.

According to a sixth aspect is provided an animal feed comprising the present enzyme composition or the recombinant host cell, and at least one protein source of plant origin or a mannan containing product or by-product, and

a. Optionally at least one enzyme selected from protease, amylase, phytase, xylanase, endoglucanase, beta-glucanase, or a combination thereof; and
b. Optionally at least one filler selected from maltodextrin, flour, salt, sodium chloride, sulfate, sodium sulfate, or a combination thereof.

According to a seventh aspect is provided a feed supplement comprising the present enzyme composition or the enzyme obtainable from host cell; and
a. Optionally at least one enzyme selected from protease, amylase, phytase, xylanase, endoglucanase, beta-glucanase, or a combination thereof; and
b. Optionally at least one filler selected from maltodextrin, flour, salt, sodium chloride, sulfate, sodium sulfate, or a combination thereof.

The feed and the feed supplement improve nutritional value of feed compared to a feed without mannanase. The present enzyme composition degrades mannan present in the feed and thereby makes it more easily digestible for the animal. In particular for soybean meal containing feeds mannan-oligosaccharides that result from enzymatic digestion have a beneficial effects on the intestinal microbes, and consequently on the performance of the animals. The effect of mannanases can be enhanced by including xylanase to digest arabinoxylans present in corn soybean based diets. Mannanase can also be used to modify rheological properties of wet feeds.

In an embodiment the feed may comprise animal protein, such as meat meal or bone meal.

According to a eighth aspect is provided a use, and a method of using, the animal feed of the sixth aspect or the feed supplement of the seventh aspect in:

a. feeding animals, preferably monogastric animals and ruminants;

b. improving weight gain of animals.

According to an ninth aspect is provided a use of, and a method of using, the present enzyme composition or the enzyme obtainable from the host cell in a detergent.

In one embodiment of the present disclosure the detergent composition further comprises one or more additional enzymes selected from the group consisting of protease, lipase, cutinase, amylase, carbohydrase, cellulase, pectinase, pectatelyase, pectinolytic enzyme, esterase, mannanase, arabinase, galactanase, xylanase, oxidase, xanthanase, xyloglucanase, laccase, DNAse and/or peroxidase, preferably selected from the group consisting of proteases, amylases, cellulases and lipases.

In a further embodiment of the present disclosure the detergent composition is in a form of a bar, a homogenous tablet, a tablet having two or more layers, a pouch having one or more compartments, a regular or compact powder, a granule, a paste, a gel, or a regular, compact or concentrated liquid. In one embodiment the detergent composition can be a laundry detergent composition, preferably a liquid or solid laundry detergent composition.

The aspects of the disclosed embodiments furthermore relate to the use of the enzyme composition or the detergent composition as herein disclosed for degrading mannan.

In a further embodiment the present disclosure relates to the use of the enzyme composition or the detergent composition as herein disclosed in a laundry process.

The aspects of the disclosed embodiments furthermore relate to a method for removing a stain from a surface, comprising contacting the surface with the enzyme composition or the detergent composition as herein disclosed.

The present disclosure also relates to a method for degrading mannan comprising applying the enzyme composition or the detergent composition as herein disclosed to mannan, preferably wherein the mannan is on a surface of a textile, or at least partially embedded in a textile.

According to a tenth aspect is provided a use of, and a method of using, the present enzyme composition of the first aspect or the enzyme obtainable from the host cell of the third aspect in oil drilling.

The present enzyme composition is advantageous in modifying rheological properties of oil drilling fluids and to improve oil recovery.

According to an eleventh aspect is provided a use of, and a method of using, the present enzyme composition of the first aspect or the enzyme obtainable from the host cell of the third aspect in processing coffee extract, fruit juice, pineapple juice, or soya milk.

Using the present enzyme composition or the enzyme obtainable from the host cell is advantageous in processing coffee extract because it reduces viscosity of the coffee extract.

Using the present enzyme composition or the enzyme obtainable from the host cell is advantageous in processing and manufacturing fruit juice because it lowers viscosity and improves filtration rate, stability and helps to extract fruit components.

Using the present enzyme composition or the enzyme obtainable from the host cell is advantageous in processing and manufacturing soya milk because it improves yield, colour, protein content and taste of soya milk.

In another aspect the disclosed sequence information herein relating to a polynucleotide sequence encoding a mannanase of the aspects of the disclosed embodiments can be used as a tool to identify other homologous mannanases. For instance, polymerase chain reaction (PCR) can be used to amplify sequences encoding other homologous mannanases from a variety of biological sources. In addition, genome mining approaches can be used to identify sequences encoding other homologous mannanases from genome databases.

### BRIEF DESCRIPTION OF THE FIGURES

FIG. 1 shows schematic representation of vector pEV1 for replication in Bacillus.

FIG. 2 schematically shows the expression cassettes used in the transformation of Trichoderma reesei protoplasts for overproducing the recombinant mannanase proteins (Man6, Man7 and Man14). The mannanase genes were under the control of T. reesei cel7A/cbh1 promoter (pcbh1) and the termination of the transcription was ensured by using T. reesei cel7A/cbh1 terminator sequence (tcbh1). The amdS gene was included as a transformation marker.

FIG. 3 describes the effect of pH on the activity of recombinant Man6, Man7 and Man14 (Bacillus produced) mannanase proteins in 40 mM Britton-Robinson buffer at pH 4 to pH 11. Reaction temperature was 50° C. and the reaction time was 10 min. Azurine-crosslinked carob galactomannan was used as a substrate. All measurements were made at least duplicates. The data points are averages of separate measurements.

FIG. 4 shows the temperature profile of recombinant Man6, Man7 and Man14 (Bacillus produced) mannanases assayed in 40 mM Britton-Robinson buffer pH 7 using 10 min reaction time, Azurine-crosslinked carob galactomannan was used as a substrate. All measurements were made at least duplicates. The data points are averages of separate measurements.

FIG. 5 shows SDS PAGE analysis of bacterial mannanases.

FIG. 6 describes the stain removal performance of Man6 and Man7 (produced in Bacillus and Trichoderma) as an increase of lightness (sum of ΔL*of 4 stains) in the presence of 4.4 g/l of Commercial heavy duty liquid detergent A at 40° C., 16° dH, 60 min, pH approx. 8.3 and enzymes dosed as activity units. Commercial preparation Mannaway® 4.0 L was used for comparison.

FIG. 7 describes the stain removal performance of Man6 and Man7 (produced in Bacillus) as an increase of lightness (sum of ΔL*of 4 stains) in the presence of 4.4 g/l of Commercial heavy duty liquid detergent A at 40° C., 16° dH, 60 min, pH approx. 8.3 and enzymes dosed as active enzyme protein (AEP). Commercial preparation Mannaway® 4.0 L was used for comparison.

FIG. 8 describes the stain removal performance of Man6 and Man7 (produced in Bacillus) as an increase of lightness (sum of ΔL*of 4 stains) in the presence of 3.8 g/l of Commercial color detergent powder at 40° C., 16° dH, 60 min, pH approx. 10 and enzymes dosed as activity units. Commercial preparation Mannaway® 4.0 L was used for comparison.

FIG. 9 describes the stain removal performance of Man6 and Man7 (produced in Bacillus) as an increase of lightness (sum of ΔL*of 4 stains) in the presence of 3.8 g/l of Commercial color detergent powder at 40° C., 16° dH, 60 min, pH approx. 10 and enzymes dosed as active enzyme protein. Commercial preparation Mannaway® 4.0 L was used for comparison.

FIG. 10 describes the stain removal performance of Man6 and Man7 (produced in Bacillus) as an increase of lightness (sum of ΔL* of 3 stains) in the presence of 4.2 g/l of Commercial bleach detergent powder at 40° C., 16° dH, 60 min, pH approximately 9.5 and enzymes dosed as active enzyme protein. Commercial preparation Mannaway® 4.0 L was used for comparison.

FIG. 11 describes the stain removal performance of Man14 (produced in Bacillus) as an increase of lightness (sum of ΔL*of 2 stains) in the presence of 5 g/l of Commercial heavy duty liquid detergent B at 40° C., 16° dH, 60 min, pH approximately 8.3 and enzymes dosed as activity units. Commercial preparation Mannaway® 4.0 L was used for comparison.

FIG. 12 describes the stain removal performance of Man14 (produced in Bacillus) as an increase of lightness (sum of ΔL*of 2 stains) in the presence of 5 g/l of Commercial heavy duty liquid detergent B at 40° C., 16° dH, 60 min, pH approximately 8.3 and enzymes dosed as active enzyme protein. Commercial preparation Mannaway® 4.0 L was used for comparison.

FIG. 13 describes the stability of Man6 and Man7 (produced in Bacillus) in liquid detergent (OMO Color) at 37° C. Commercial preparation Mannaway® 4.0 L was used for comparison

FIG. 14 describes the stability of Man7 (produced both in Bacillus and Trichoderma) and Man6 (produced in Bacillus) in Commercial heavy duty liquid detergent A. Commercial preparation Mannaway® 4.0 L was used for comparison.

FIG. 15 shows a flow chart of instant coffee production involving use of the mannanase of the aspects of the disclosed embodiments.

### SEQUENCE LISTINGS

SEQ ID NO: 1 Sequence of the oligonucleotide primer Man6_1

SEQ ID NO: 2 Sequence of the oligonucleotide primer Man6_2

SEQ ID NO: 3 Sequence of the oligonucleotide primer Man7_1

SEQ ID NO: 4 Sequence of the oligonucleotide primer Man7_2

SEQ ID NO: 5 Sequence of the oligonucleotide primer Man14_1

SEQ ID NO: 6 Sequence of the oligonucleotide primer Man14_2

SEQ ID NO: 7 Sequence of the oligonucleotide primer Vec_1

SEQ ID NO: 8 Sequence of the oligonucleotide primer Vec_2

SEQ ID NO: 9 The nucleotide sequence of the Bacillus clausii man6

SEQ ID NO: 10 The nucleotide sequence of the Bacillus clausii man6 without signal peptide encoding sequence and with codon optimization to Trichoderma reesei

SEQ ID NO: 11 The deduced amino acid sequence of the Bacillus clausii Man6

SEQ ID NO: 12 The deduced amino acid sequence of the Bacillus clausii Man6 without signal peptide

SEQ ID NO: 13 The nucleotide sequence of the Bacillus hemicellulosilyticus man7

SEQ ID NO: 14 The nucleotide sequence of the Bacillus hemicellulosilyticus man7 without signal peptide encoding sequence and with codon optimization to Trichoderma reesei

SEQ ID NO: 15 The deduced amino acid sequence of the Bacillus hemicellulosilyticus Man7

SEQ ID NO: 16 The deduced amino acid sequence of the Bacillus hemicellulosilyticus Man7 without signal peptide

SEQ ID NO: 17 The nucleotide sequence of the Virgibacillus soli man14

SEQ ID NO: 18 The nucleotide sequence of the Virgibacillus soli man14 without signal peptide encoding sequence and with codon optimization to Trichoderma reesei

SEQ ID NO: 19 The deduced amino acid sequence of the Virgibacillus soli Man14

SEQ ID NO: 20 The deduced amino acid sequence of the Virgibacillus soli Man14 without signal peptide

SEQ ID NO: 21 Sequence of the oligonucleotide primer BMAN1

SEQ ID NO: 22 Sequence of the oligonucleotide primer BMAN2

SEQ ID NO: 23 Sequence of the oligonucleotide primer BMAN3

SEQ ID NO: 24 Sequence of the oligonucleotide primer BMAN4

SEQ ID NO: 25 The nucleotide sequence of Bacillus pumilus man31

SEQ ID NO: 26 The deduced amino acid sequence of the Bacillus pumilus Man31

SEQ ID NO: 27 The nucleotide sequence of the Bacillus amyloliquefaciens man32

SEQ ID NO: 28 The deduced amino acid sequence of the Bacillus amyloliquefaciens Man32

SEQ ID NO: 29 The nucleotide sequence of the Amphibacillus xylanus man33

SEQ ID NO: 30 The deduced amino acid sequence of the Amphibacillus xylans Man33

SEQ ID NO: 31 The nucleotide sequence of the Paenibacillus polymyxa man34

SEQ ID NO: 32 The deduced amino acid sequence of the Paenibacillus polymyxa Man34

SEQ ID NO: 33 The nucleotide sequence of the Bacillus hemicellulosilyticus man35

SEQ ID NO: 34 The deduced amino acid sequence of the Bacillus hemicellulosilyticus Man35

SEQ ID NO: 35 The nucleotide sequence of the Bacillus alcalophilus man36

SEQ ID NO: 36 The deduced amino acid sequence of the Bacillus alcalophilus Man36

SEQ ID NO: 37 The nucleotide sequence of the Bacillus sp. man37

SEQ ID NO: 38 The deduced amino acid sequence of the Bacillus sp. Man37

SEQ ID NO: 39 The nucleotide sequence of the Bacillus circulans man38

SEQ ID NO: 40 The deduced amino acid sequence of the Bacillus circulans Man38

SEQ ID NO: 41 The nucleotide sequence of the Paenibacillus sp. man39

SEQ ID NO: 42 The deduced amino acid sequence of the Paenibacillus sp. Man39

SEQ ID NO: 43 The nucleotide sequence of the Bacillus circulans man40

SEQ ID NO: 44 The deduced amino acid sequence of the Bacillus circulans Man40

SEQ ID NO: 45 The nucleotide sequence of the Bacillus nealsonii man41

SEQ ID NO: 46 The deduced amino acid sequence of the Bacillus nealsonii Man41

SEQ ID NO: 47 The nucleotide sequence of the Bacillus circulans man42

SEQ ID NO: 48 The nucleotide sequence of the Bacillus circulans Man42

### DETAILED DESCRIPTION

Mannan refers to polysaccharides consisting of a mannose backbone linked together by β-1,4-linkages with side-chains of galactose attached to the backbone by α-1,6-linkages. Mannans comprise plant-based material such as guar gum and locust bean gum. Glucomannans are polysaccharides having a backbone of more or less regularly alternating β-1,4 linked mannose and glucose, galactomannans and galactoglucomannans are mannans and glucomannans with alpha-1,6 linked galactose side branches.

As used herein, the term “mannanase” or “galactomannanase” denotes a mannanase enzyme defined according to that known in the art as mannan endo-1,4-beta-mannosidase and having the alternative names beta-mannanase and endo-1,4-mannanase and catalysing hydrolysis of 1,4-beta-D-mannosidic linkages in mannans, galactomannans, glucomannans, and galactoglucomannans. Mannanases are classified according to the Enzyme Nomenclature as EC 3.2.1.78.

As used herein, “isolated” means a substance in a form or environment that does not occur in nature. Non-limiting examples of isolated substances include (1) any non-naturally occurring substance, (2) any substance including any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing or decreasing the amount of the substance relative to other components with which it is naturally associated (e.g., recombinant production in a host cell; one or multiple copies of a gene encoding the substance; and use of an alternative promoter to the promoter naturally associated with the gene encoding the substance). In an embodiment a ss polypeptide, enzyme, polynucleotide, host cell or composition of the present disclosure is isolated.

As used herein, the term “comprising” includes the broader meanings of “including”, “containing”, and “comprehending”, as well as the narrower expressions “consisting of” and “consisting only of”.

As used herein, “fragment” means a protein or a polynucleotide having one or more amino acids or nucleotides deleted. In the context of DNA, a fragment includes both single-stranded and double-stranded DNA of any length. A fragment may be an active fragment, which has the biological function, such as enzyme activity or regulatory activity, of the protein or the polynucleotide. A fragment may also be an inactive fragment, i.e. it does not have one or more biological effects of the native protein or polynucleotide.

As used herein, a “peptide” and a “polypeptide” are amino acid sequences including a plurality of consecutive polymerized amino acid residues. For purpose of the aspects of the disclosed embodiments, peptides are molecules including up to 20 amino acid residues, and polypeptides include more than 20 amino acid residues. The peptide or polypeptide may include modified amino acid residues, naturally occurring amino acid residues not encoded by a codon, and non-naturally occurring amino acid residues. As used herein, a “protein” may refer to a peptide or a polypeptide of any size. A protein may be an enzyme, a protein, an antibody, a membrane protein, a peptide hormone, regulator, or any other protein.

The term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

As used herein, “modification”, “modified”, and similar terms in the context of polynucleotides refer to modification in a coding or a non-coding region of the polynucleotide, such as a regulatory sequence, 5′ untranslated region, 3′ untranslated region, up-regulating genetic element, down-regulating genetic element, enhancer, suppressor, promoter, exon, or intron region. The modification may in some embodiments be only structural, having no effect on the biological effect, action or function of the polynucleotide. In other embodiments the modification is a structural modification, which provides a change in the biological effect, action or function of the polynucleotide. Such a modification may enhance, suppress or change the biological function of the polynucleotide.

As used herein, “identity” means the percentage of exact matches of amino acid residues between two aligned sequences over the number of positions where there are residues present in both sequences. When one sequence has a residue with no corresponding residue in the other sequence, the alignment program allows a gap in the alignment, and that position is not counted in the denominator of the identity calculation. Identity is a value determined with the Pairwise Sequence Alignment tool EMBOSS Needle at the EMBL-EBI website (www.ebi.ac.uk/Tools/psa/emboss_needle/).

As used herein, “host cell” means any cell type that is susceptible to transformation, transfection, transduction, mating, crossing or the like with a nucleic acid construct or expression vector comprising a polynucleotide. The term “host cell” encompasses any progeny that is not identical due to mutations that occur during replication. Non-limiting examples of a host cell are fungal cells, filamentous fungal cells from Division Ascomycota, Subdivision Pezizomycotina; preferably from the group consisting of members of the Class Sordariomycetes, Subclass Hypocreomycetidae, Orders Hypocreales and Microascales and Aspergillus, Chrysosporium, Myceliophthora and Humicola; more preferably from the group consisting of Families Hypocreacea, Nectriaceae, Clavicipitaceae, Microascaceae, and Genera Trichoderma (anamorph of Hypocrea), Fusarium, Gibberella, Nectria, Stachybotrys, Claviceps, Metarhizium, Villosiclava, Ophiocordyceps, Cephalosporium, and Scedosporium; more preferably from the group consisting of Trichoderma reesei (Hypocrea jecorina), T. citrinoviridae, T. longibrachiatum, T. virens, T. harzianum, T. asperellum, T. atroviridae, T. parareesei Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Acremonium (Cephalosporium) chrysogenum, and Scedosporium apiospermum, and Aspergillus niger, Aspergillus awamori, Aspergillus oryzae, Chrysosporium lucknowense, Myceliophthora thermophila, Humicola insolens, and Humicola grisea, most preferably Trichoderma reesei. Non-limiting examples of a host cell are bacterial cells, preferably gram positive Bacilli (e.g. Bacillus subtilis, B. licheniformis, B. megaterium, B. amyloliquefaciens, B. pumilus), gram-negative bacteria (e.g. Escherichia coli), actinomycetales (e.g. Streptomyces sp.) and yeasts (e.g. Saccharomyces cerevisiae, Pichia pastoris, Yarrowia lipolytica).

In an embodiment the host cell is a fungal cell, preferably a filamentous fungal cell, such as Trichoderma or Trichoderma reesei. In an embodiment the host cell is a bacterial cell, preferably a gram positive Bacillus cell, such as B. subtilis, B. licheniformis, B. megaterium, B. amyloliquefaciens, B. pumilus.

A “recombinant cell” or “recombinant host cell” refers to a cell or host cell, which has been genetically modified or altered to comprise a nucleic acid sequence which is not native to said cell or host cell. In an embodiment the genetic modification comprises integrating the polynucleotide in the genome of the host cell. In another embodiment the polynucleotide is exogenous in the host cell.

As used herein, “expression” includes any step involved in the production of a polypeptide in a host cell including, but not limited to, transcription, translation, post-translational modification, and secretion. Expression may be followed by harvesting, i.e. recovering, the host cells or the expressed product.

The term “expression vector” denotes a DNA molecule, linear or circular, that comprises a segment encoding a polypeptide of interest operably linked to additional segments that provide for its transcription. Such additional segments may include promoter and terminator sequences, and may optionally include one or more origins of replication, one or more selectable markers, an enhancer, a polyadenylation signal, carrier and the like. Expression vectors are generally derived from plasmid or viral DNA, or may contain elements of both. The expression vector may be any expression vector that is conveniently subjected to recombinant DNA procedures, and the choice of vector will often depend on the host cell into which the vector is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The term “recombinant produced” or “recombinantly produced” used herein in connection with production of a polypeptide or protein is defined according to the standard definition in the art.

The term “obtained from” and “obtainable” as used herein in connection with a specific microbial source means that the polynucleotide is expressed by the specific source (homologous expression), or by a cell in which a gene from the source has been inserted (heterologous expression).

The term “enzyme composition” means either a conventional enzymatic fermentation product, possibly isolated and purified, from a single species of a microorganism, such preparation usually comprising a number of different enzymatic activities; or a mixture of monocomponent enzymes, preferably enzymes derived from bacterial or fungal species by using conventional recombinant techniques, which enzymes have been fermented and possibly isolated and purified separately and which may originate from different species, preferably fungal or bacterial species or the fermentation product of a microorganism which acts as a host cell for production of a recombinant mannanase, but which microorganism simultaneously produces other enzymes.

The term “operably linked”, when referring to DNA segments, denotes that the segments are arranged so that they function in concert for their intended purposes, e.g. transcription initiates in the promoter and proceeds through the coding segment to the terminator

The term “promoter” denotes a portion of a gene containing DNA sequences that provide for the binding of RNA polymerase and initiation of transcription. Promoter sequences are commonly, but not always, found in the 5′ non-coding regions of genes.

The term “secretory signal sequence” denotes a DNA sequence that encodes a polypeptide (a “secretory peptide”) that, as a component of a larger polypeptide, directs the larger polypeptide through a secretory pathway of a host cell in which it is produced. The secretory signal sequence can be native or it can be replaced with secretory signal sequence or carrier sequence from another source. Depending on the host cell, the larger peptide may be cleaved to remove the secretory peptide during transit through the secretory pathway.

The term “core region” denotes a domain of an enzyme, which may or may not have been modified or altered, but which has retained at least part of its original activity; the catalytic domain as known in the art has remained functional. The core region of a mannanase according to the aspects of the disclosed embodiments correspond to the amino acids aligned with the amino acids 27-331 of Man7, SEQ ID NO: 16, amino acids 35-324 of Man6, SEQ ID NO: 12, or amino acids 17-314 of Man14, SEQ ID NO: 20.

By the term “linker” or “spacer” is meant a polypeptide comprising at least two amino acids which may be present between the domains of a multidomain protein, for example an enzyme comprising an enzyme core and a binding domain such as a carbohydrate binding module (CBM) or any other enzyme hybrid, or between two proteins or polypeptides produced as a fusion polypeptide, for example a fusion protein comprising two core enzymes. For example, the fusion protein of an enzyme core with a CBM is provided by fusing a DNA sequence encoding the enzyme core, a DNA sequence encoding the linker and a DNA sequence encoding the CBM sequentially into one open reading frame and expressing this construct.

Efficient amount means an amount, which is sufficient to degrade mannose in the selected application.

The terms “detergent composition” and “detergent” include, unless otherwise indicated, solid, granular or powder-form all-purpose or heavy-duty washing agents, especially cleaning detergents; liquid, gel or paste-form all-purpose washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, car or carpet shampoos, bathroom cleaners; metal cleaners; as well as cleaning auxiliaries such as bleach additives and “stain-stick” or pre-treat types. The terms “detergent”, “detergent composition” and “detergent formulation” are used in reference to mixtures, which are intended for use in a wash medium for the cleaning of soiled objects. In some embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present disclosure be limited to any particular detergent formulation or composition. It is intended that in addition to the mannanases according to the aspects of the disclosed embodiments, the term encompasses detergents that may contain e.g., surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anticorrosion agents, hydrotropes, fabric hueing agents, dispersants, dye transfer inhibiting agents, fluorescent whitening agents, soil release polymers, anti-redepositions agents, anti-shrink agents, anti-wrinkling agents, bactericides, binders, carriers, dyes, enzyme stabilizers, fabric softeners, fillers, foam regulators, perfumes, pigments, sod suppressors, solvents, and structurants for liquid detergents, structure elasticizing agents, enzyme inhibitors or stabilizers, enzyme activators, transferase(s), hydrolytic enzymes, oxido reductases, bluing agents and fluorescent dyes, antioxidants, and solubilizers.

The term “textile” means any textile material including yarns, yarn intermediates, fibers, non-woven materials, natural materials, synthetic materials, and any other textile material, fabrics made of these materials and products made from fabrics (e.g., garments, linen and other articles). The textile or fabric may be in the form of knits, wovens, denims, non-wovens, felts, yarns, and towelling. The textile may be cellulose based, such as natural cellulosics including cotton, flax/linen, jute, ramie, sisal or coir or manmade cellulosics (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabbit and silk or synthetic polymer such as nylon, aramid, polyester, acrylic, polypropylene and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). Fabric may be conventional washable laundry, for example stained household laundry. When the term fabric or garment is used it is intended to include the broader term textiles as well.

The term “stability” includes storage stability and stability during use, e.g. during a wash process (in wash stability) and reflects the stability of the mannanase according to the aspects of the disclosed embodiments as a function of time, e.g. how much activity is retained when the mannanase is kept in solution, in particular in a detergent solution. The stability is influenced by many factors, e.g. pH, temperature, detergent composition e.g. proteases, stabilizers, builders, surfactants etc. The mannanase stability may be measured using the ‘activity assay’ as described in examples.

“Mannanase activity” as used herein refers to the mannan degrading activity of a polypeptide. Degrading or modifying as used herein means that mannose units are hydrolyzed from the mannan polysaccharide by the mannanase. The mannan degrading activity of the polypeptides according to present disclosure can be tested according to standard test procedures known in the art. Example 7 provides an example of a standard method for determining mannanase activity.

In a further embodiment of the present disclosure the at least one enzyme has mannanase activity. The mannanases comprised in the present enzyme composition of the aspects of the disclosed embodiments are suitable for degrading and modifying mannan containing material in various chemical environments, preferably in detergent compositions.

In one embodiment of the present disclosure the enzyme composition further comprises one or more additional enzymes selected from the group consisting of protease, lipase, cutinase, amylase, carbohydrase, cellulase, pectinase, pectatelyase, pectinolytic enzyme, esterase, phytase, mannanase, arabinase, galactanase, xylanase, oxidase, xanthanase, xyloglucanase, DNAse, laccase, and/or peroxidase, preferably selected from the group consisting of proteases, amylases, cellulases and lipases.

The present enzyme composition comprising mannanase and an additional enzyme is advantageous in providing synergistic effect. Such additional enzymes are desired when the present enzyme composition comprising mannanase is used in detergents e.g. when washing stains. Particularly advantageous synergistic enzymes that work with mannanase are amylases, proteases and cellulases, or a combination thereof, such as a composition comprising mannanase, amylase and protease.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

A composition for use in solid laundry detergent, for example, may include 0.000001%-5%, such as 0.000005-2%, such as 0.00001%-1%, such as 0.00001%-0.1% of enzyme protein by weight of the composition.

A composition for use in laundry liquid, for example, may include 0.000001%-3%, such as 0.000005%-1%, such as 0.00001%-0.1% of enzyme protein by weight of the composition.

A composition for use in automatic dishwash, for example, may include 0.000001%-5%, such as 0.000005%-2%, such as 0.00001%-1%, such as 0.00001%-0.1% of enzyme protein by weight of the composition.

In a further embodiment of the present disclosure the detergent composition is in the form of a bar, a homogenous tablet, a tablet having two or more layers, a pouch having one or more compartments, a regular or compact powder, a granule, a paste, a gel, or a regular, compact or concentrated liquid. In one embodiment the detergent composition can be a laundry detergent composition, preferably a liquid or solid laundry detergent composition. There are a number of detergent formulation forms such as layers (same or different phases), pouches, as well as forms for machine dosing unit.

In an embodiment the present enzyme composition further comprises:

a. at least one preservative selected from benzoic acid, sodium benzoate, hydroxybenzoate, citric acid, ascorbic acid, or a combination thereof;
b. optionally at least one polyol selected from propylene glycol, glycerol, a sugar, sugar alcohol, lactic acid, boric acid, boric acid derivative, aromatic borate ester, phenyl boronic acid derivative, peptide, or a combination thereof;
c. optionally at least one enzyme selected from proteases, amylases, cellulases, lipases, xylanases, mannanases, cutinases, esterases, phytases, DNAses, pectinases, pectinolytic enzymes, pectate lyases, carbohydrases, arabinases, galactanases, xanthanases, xyloglucanase, laccases, peroxidases and oxidases with or without a mediator, or a combination thereof; and
d. optionally at least one filler selected from maltodextrin, flour, sodium chloride, sulfate, sodium sulfate, or a combination thereof.

The additional components a-d provide improved properties for the present enzyme composition. The enzyme composition is compatible with the additional components and improves applicability of the enzyme composition in various uses.

Salts, such as sodium chloride and sodium sulfate function as drying aids.

In an embodiment of the first aspect the present enzyme composition is in the form of a liquid composition or a solid composition such as solution, dispersion, paste, powder, granule, granulate, coated granulate, tablet, cake, crystal, crystal slurry, gel or pellet.

The present disclosure furthermore relates to different uses of the enzyme composition as herein disclosed, such as for degrading mannan and for use in a laundry process.

An enzyme composition can also be used in cleaning agents or boosters that are added on top of the detergent during or before the wash and that are for example in the form of liquid, gel, powder, granules or tablets. Enzyme composition and detergent components may also be soaked in a carrier like textiles.

In an embodiment the mannanase has relative activity of at least 50% in the pH range from 5.5 to 8.5. The relative activity may be determined by the method described in Example 7.

In an embodiment of the present disclosure the mannanase has a relative activity of at least 30% in the temperature range from 45° to 65° C.

Providing mannanases that retain activity in temperatures above ambient temperature is advantageous for applications wherein mannan degradation is required in such conditions. Further, the mannanases according to the aspects of the disclosed embodiments may have good stability and activity in alkaline conditions, which is advantageous in detergent use and in biomass processing.

In an embodiment the mannanase enzyme has an amino acid sequence with at least or about 93%, 94%, 95%, 96%, 97%, 98%, 99% or 100% sequence identity to SEQ ID NO: 12.

In an embodiment the mannanase enzyme has an amino acid sequence with at least or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:16.

In an embodiment the mannanase enzyme has an amino acid sequence with at least or about 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 20.

In an embodiment the mannanase enzyme has an amino acid sequence which is not 100% identical to SEQ ID NO: 12 [Man6], SEQ ID NO: 16 [Man7], or SEQ ID NO: 20 [Man14].

In an embodiment the present enzyme composition comprises the recombinant host cell of the second aspect.

In an embodiment of the second aspect the recombinant the recombinant polypeptide is a fusion protein which, in addition to having the amino acid sequence having mannanase activity, comprises at least one of:

an amino acid sequence providing a secretory signal sequence, such as Bacillus amyloliquefaciens xylanase signal sequence;

an amino acid sequence which facilitates purification, such as an affinity tag, His-tag;
an amino acid sequence which enhances production, such as an amino acid sequence which is a carrier, such as CBM;
an amino acid sequence having an enzyme activity; and
an amino acid sequence providing for the fusion protein with binding affinity, such as a carbohydrate binding moiety.

The CBM, carbohydrate binding moiety, as a carrier is advantageous e.g. in Trichoderma production.

In an embodiment the host cell is non-pathogenic. This is particularly advantageous for using the host cell in feed, and in detergent applications such as in home laundry detergents.

In an embodiment of the fifth aspect the mannan containing material is selected from plant based material, textile, waste water, sewage, oil or a combination thereof.

In another embodiment the mannan containing material is recycled waste paper; mechanical pulp, chemical pulp, semi chemical pulp, Kraft or other paper-making pulps; fibres subjected to a retting process; or guar gum or locust bean gum containing material.

In another embodiment degradation or modifying is carried out in an aqueous environment wherein mannanase shows activity.

In a preferred embodiment the mannan containing material, which is degraded or modified in the method, is on a textile or a fabric optionally with mannan stains. By degrading mannan attached to the textile or fabric, dirt or soil bound to mannan is released and not capable of binding again to the mannan or mannan stains. The textile or fabric can be of any material, for example cotton, flax/linen, jute, ramie, sisal or coir or manmade cellulosics (e.g. originating from wood pulp) including viscose/rayon, modal, cellulose acetate fibers (tricell), lyocell, cupro or blends thereof.

In an embodiment of the sixth aspect the animal is a monogastric animal or a ruminant. In another embodiment the animal is a broiler chicken, egg-laying chicken, swine, turkey, or an aquaculture organism such as fish. In another embodiment the animal is a ruminant.

In an embodiment the feed comprises or consists of maize and soybean meal.

In an embodiment the protein source of plant origin comprises or consist of soy, cereal such as barley, wheat, rye, oats, or maize.

In an embodiment the mannan containing product or by-product comprises or consists of palm kernel, guar meal or copra meal.

In an embodiment of the sixth or seventh aspect the animal feed or the feed supplement is formulated in the form of a wet composition or a dry composition.

In an embodiment or the ninth aspect the detergent is a liquid detergent or a solid detergent preferably in a form of a powder, bar, tablet, pouch, paste, gel, liquid, granule or granulate.

In an embodiment the composition comprising at least one mannanase enzyme is used in pulp and paper industry, biobleaching, fiber modification, drainage improvement and in the oil industry, i.e. in oil drilling or oil-servicing industry for hydro-fracturing or controlling the viscosity of drilling fluids.

In an embodiment the composition comprising at least one mannanase enzyme is used in textile and detergent industry, biomass processing and biomass hydrolysis, preferably in biofuel, starch, pulp and paper, food, baking, feed or beverage industries.

In an embodiment the mannanase hydrolyses endo-beta-1,4-mannosidic linkages randomly.

In an embodiment the mannanase is obtainable or derivable from a bacterial source.

In an embodiment the mannanase can be fused with at least one further polypeptide, thus forming a fusion polypeptide. The fusion polypeptide or the further polypeptide may have other catalytic or binding activities in addition to those of mannanase. In an embodiment the further polypeptide comprises or consists of carbohydrate binding module, which is optionally a fragment of another protein or enzyme derived from the same or different organism as the mannanase.

In an embodiment the mannanase is connected to the further polypeptide with a linker.

In an embodiment is provided a process for machine treatment of fabrics which process comprises treating fabric during a washing cycle of a machine washing process with a washing solution containing the enzyme composition of the first aspect, the enzyme obtainable from the recombinant host cell of the second aspect or the recombinant polypeptide of the third aspect.

In an embodiment is provided a use of the enzyme composition of the first aspect, the enzyme obtainable from the recombinant host cell of the second aspect, or the polypeptide of the third aspect together with an enzyme selected from protease, amylase, cellulase, lipase, xylanase, mannanase, cutinase, esterase, phytase, DNAse, pectinase, pectinolytic enzyme, pectate lyase, carbohydrase, arabinase, galactanase, xanthanase, xyloglucanase, laccase, peroxidase and oxidase with or without a mediator in a cleaning composition for fabric cleaning and/or fabric stain removal.

In an embodiment is provided a use of the enzyme composition of the first aspect, the enzyme obtainable from the recombinant host cell of the second aspect, or the polypeptide of the third aspect together with an enzyme selected from protease, amylase, cellulase, lipase, xylanase, mannanase, cutinase, esterase, phytase, DNAse, pectinase, pectinolytic enzyme, pectate lyase, carbohydrase, arabinase, galactanase, xanthanase, xyloglucanase, laccase, peroxidase and oxidase with or without a mediator in a cleaning composition for cleaning hard surfaces such as floors, walls, bathroom tile and the like.

In an embodiment is provided a use of the enzyme composition of the first aspect, the enzyme obtainable from the recombinant host cell of the second aspect, or the polypeptide of the third aspect together with an enzyme selected from protease, amylase, cellulase, lipase, xylanase, mannanase, cutinase, esterase, phytase, DNAse, pectinase, pectinolytic enzyme, pectate lyase, carbohydrase, arabinase, galactanase, xanthanase, xyloglucanase, laccase, peroxidase and oxidase with or without a mediator in a cleaning composition for hand and machine dishwashing.

### EXAMPLES

The following examples are provided to illustrate various aspects of the present disclosure. They are not intended to limit the aspects of the disclosed embodiments, which is defined by the accompanying claims.

### Example 1. Screening

For identification of new beta-1,4-mannanases public databases (NCBI, EBI) and selected proprietary and public genomes were screened. All proprietary and public genomes used in this work are shown in Table 1. All hits were grouped and finally 15 genes of bacterial origin were selected for cloning in Bacillus based on the phylogenetic distance between each other (Table 2)

TABLE 1
List of proprietary and public genomes used
for screening of beta-1,4-mannanases

| Species | Strain | Source |
|---|---|---|
| Bacillus pumilus | MS8 | ABE |
| Amphibacillus xylanus | NBRC 15112 | NCBI |
| Bacillus hemicellulosilyticus | JCM 9152 | NCBI |
| Bacillus clausii | KSM-K16 | NCBI |
| Bacillus amyloliquefaciens | RH1330 | ABE |
| Virigibacillus soli | PL205 | NCBI |

TABLE 2
List of genes selected for cloning in Bacillus. Predicted PFAM domains
and amino acid lengths of the proteins are shown

| Sequence ID | Species | GH family | Length |
|---|---|---|---|
| orf2511 | Bacillus amyloliquefaciens | 26 | 360 aa |
| AXY_08250 | Amphibacillus xylanus | 5 | 497 aa |
| man7 | Bacillus hemicellulosilyticus | 5 | 490 aa |
| T1Z249.2 | Bacillus nealsonii | 5 | 369 aa |
| man6 | Bacillus clausii | 5 | 324 aa |
| Q9EYQ3 | Clostridium cellulolyticum | 5 | 424 aa |
| YdhT | Bacillus cellulosilyticus | 26 | 1183 aa |
| V5X1N9 | Paenibacillus polymyxa | 5 | 588 aa |
| Q9ZI87 | Geobacillus stearothermophilus | 5 | 694 aa |
| Q49HI4 | Bacillus circulans | 5 | 327 aa |
| orf0659 | Bacillus pumilus | 5 | 376 aa |
| JCM9152_1090 | Bacillus hemicellulosilyticus | 26 | 489 aa |
| D3HC62 | Streptococcus gallolyticus | 5 | 487 aa |
| A0LSH9 | Acidothermus cellulolyticus | 5 | 763 aa |
| man14 | Virgibacillus soli | 5 | 482 aa |

### Example 2. Cloning of Bacterial Mannanases in Bacillus

Unless otherwise stated, the molecular biological methods including DNA manipulations and transformations were performed as described in Sambrook and Russell (2001) and Harwood and Cutting (1990). The genes man6, man7 and man14 were amplified by PCR using Pfx Accu Prime Polymerase (Invitrogen). PCRs were performed according to manufacturer's instructions. Following PCR conditions were used for construction of the expression plasmids: 120 sec initial denaturation at 94° C., followed by 35 cycles of 15 sec at 94° C., 30 sec annealing at one of the following 50/55° C., 110/290 sec extension at 68° C. and the final extension at 68° C. for 10 min. For amplification of man7 genomic DNA of Bacillus hemicellulosilyticus JCM 9152 was used. man6 and man14 were ordered as synthetic genes without codon optimization (Eurofins MWG, Germany). Sequences of primers used for cloning are shown in Table 3. Overhangs for hybridization are underlined.

TABLE 3
List of primers used for amplification of
man6, man7 and man14

| | | | | Seq ID |
|---|---|---|---|---|
| Template | Primer | bp | Sequence | No |
| syn. gene man6 | Man6_1 | 39 | CAACCGCCTCTGCAGCTTATGCAC | 1 |
| | | | AAAACGGATTTCACG | |
| syn. gene man6 | Man6_2 | 39 | CGGTATATCTCTGTCTTAATCACTC | 2 |
| | | | TTAAGCCCATTTTC | |
| g DNA B. | Man7_1 | 37 | CAACCGCCTCTGCAGCTTCTGATG | 3 |
| hemicellulosilyticus | | | GTCATAGCCAAAC | |
| g DNA B. | Man7_2 | 36 | CGGTATATCTCTGTCTTATTGGATT | 4 |
| hemicellulosilyticus | | | GTTACATGATC | |
| syn. Gene man14 | Man14_1 | 40 | CAACCGCCTCTGCAGCTGCAAGC | 5 |
| | | | GGGTTTTATGTAAACGG | |
| syn. Gene man14 | Man14_2 | 39 | CGGTATATCTCTGTCTTATTTAATG | 6 |
| | | | GTAACGTTATCAAC | |
| pUB110 derivate | Vec_1 | 17 | AGCTGCAGAGGCGGTTG | 7 |
| pUB110 derivate | Vec_2 | 21 | GACAGAGATATACCGACAGTG | 8 |

Genes were cloned in a standard vector pEV1 pEV1 (FIG. 1), a pUB110 derivate including promoter PaprE from Bacillus licheniformis and xylanase signal peptide from Bacillus amyloliquefaciens, by using NEBuilder® Hifi DNA Assembly Master Mix (NEB, Frankfurt). A vector:insert ration of 1:3 was applied for cloning. The total amount of fragments was at 0.2 pmol in a total volume of 20 μl. Samples were incubated for 40 min at 50° C. For construction purposes, expression plasmids were transformed by induced competence in Bacillus subtilis SCK6 as described in Zhang & Zhang 2011. The transformed cells were plated onto LB (Luria-Bertani) plates supplemented with 10 mg/l Kanamycin. Plates were incubated for 20 h at 37° C. Arising colonies were picked and plasmid was isolated using QiaPrep MiniPrep Kit (Qiagen, Hilden). Isolation procedure was carried out according to the manufacturers recommendations for Gram positives plasmid preparations. Inserts were sequenced via Sanger sequencing (GATC, Germany) and revealed the DNA sequences corresponding to the mature parts of the mannanases Man6, Man7 and Man14. Sequence comparisons were done using ClustalW sequence alignment (Thompson et al 1994). Finally, expression plasmids were transformed in an appropriate Bacillus production strain via electroporation. Bacillus production strain was grown in electroporation medium containing 20 g/l Trypton, 10 g/l yeast extract, 10 g NaCl and 2 M saccharose and 10 ml were harvested at an OD (600 nm) of 0.4. Cells were washed with electroporation buffer containing 0.272 M saccharose, 1 mM MgCl₂ and 7 mM KH₂PO₄ and finally resuspended in 250 μl electroporation buffer. Electroporation was performed using following conditions: 1.2 kV, 150 Ω, 50 μF. 1 ml electroporation medium was added afterwards and cells were incubated for 3 h at 37° C. Cells were plated on LB plates supplemented with 20 mg/l kanamycin and incubated for 18 h at 37° C. Clones were verified as described above and used for generation of material for analytic tests. Therefore, strains were inoculated in a standard expression under protein inducing conditions and incubated for 30 h at 37° C. Supernatants were harvested and used for analytical and application tests. Genes and enzyme characteristics are shown in Table 4 and 5.

TABLE 4
The summary on the GH5 family mannanase encoding genes
from Bacillus clausii KSM-K16, Bacillus hemicellulosilyticus
JCM 9152 and Virgibacillus soli PL205.

| | Length | |
|---|---|---|
| | including | |
| Gene | SP (bp) | SEQ ID NO |
| man6 | 975 | 9 |
| man7 | 1473 | 13 |
| man14 | 1449 | 17 |

TABLE 5
The summary of the amino acid sequences deduced from
the GH5 mannanase encoding gene sequences from Bacillus clausii
KSM-K16, Bacillus hemicellulosilyticus JCM 9152 and
Virgibacillus soli PL205.

| | | | | Predicted | | |
|---|---|---|---|---|---|---|
| | | | | MW (Da), | Predicted | |
| Man | No of | Length of | | ss not | pl, ss not | SEQ ID |
| protein | AAs | SS | CBM | included | included | NO |
| Man6 | 324 | 35 | | 31.84 | 4.56 | 11 |
| Man7 | 490 | 21 | Yes | 51.36 | 4.81 | 15 |
| Man14 | 482 | 16 | Yes | 50.68 | 4.35 | 19 |

### Example 3. PCR-Cloning of Bacterial Mannanases Man6 and Man7 in Trichoderma reesei

Standard molecular biology methods were used in the isolation and enzyme treatments of DNA (e.g. isolation of plasmid DNA, digestion of DNA to produce DNA fragments), in E. coli transformations, sequencing etc. The basic methods used were either as described by the enzyme, reagent or kit manufacturer or as described in the standard molecular biology handbook, e.g. Sambrook and Russell (2001). Isolation of genomic DNA was performed as described in detail by Raeder and Broda (1985).

Man6 and man7 from Bacillus clausii and Bacillus hemicellulosilyticus, respectively, were also cloned for expression in Trichoderma reesei. The genes were PCR-cloned using synthetic genes with codon optimization for Trichoderma reesei. DNA sequences encoding the signal peptides of man6 and man7 were removed by using PCR and new cloning sites created. The sequences of the primers are shown in Table 6 (SEQ ID NOs: 21-24).

TABLE 6
The oligonucleotides used as PCR primers to amplify Bacillus
hemicellulosilyticus and Bacillus clausii mannanase genes.

| Template, | | | | |
|---|---|---|---|---|
| (synthetic) DNA | Oligo- | Length | | SEQ |
| from | nucleotides | (bp) | Sequence^{(a} | ID NO: |
| Bacillus | BMAN1 | 60 | 5′-AGTCAATCGCG | 21 |
| hemicellulosilyticus | | | ACAAGCGCCAGACCC | |
| | | | ACTCGGGCTTCTACA | |
| | | | TCGAGGGCTCGACGC | |
| | | | TCTA-3′ (s) | |
| Bacillus | BMAN2 | 46 | 5′-CGCGCCGGATC | 22 |
| hemicellulosilyticus | | | CTTACTGGATCGTGA | |
| | | | CGTGGTCCAGGTAGA | |
| | | | TGGCG-3′ (as) | |
| Bacillus clausii | BMAN 3 | 60 | 5′-AGTCAATCGCG | 23 |
| | | | ACAAGCGCCAGAACG | |
| | | | GCTTCCACGTCTCCG | |
| | | | GCACGGAGCTCCTGG | |
| | | | ACAA-3′ (s) | |
| Bacillus clausii | BMAN4 | 50 | 5′-CGCGCCGGATC | 24 |
| | | | CTTAGTCGCTCTTCA | |
| | | | GGCCGTTCTCGCCGT | |
| | | | AGACGATGCG-3′ (as) | |

^{(a}″s″ in the parenthesis = sense strand, ″as″ = antisense strand.

The genes were amplified by PCR with primers described in Table 6 and using synthetic DNAs as templates in the reactions. The PCR mixtures of Bacillus clausii man6 and Bacillus hemicellulosilyticus man7 contained each 1×HF buffer for Phusion HF Polymerase (NEB/BioNordika, Finland), 0.2 mM dNTP mix (Thermo Fisher Scientific, Finland), 1 μM each primer, 3% DMSO (Thermo Fisher Scientific), 1 unit of Phusion High-Fidelity Polymerase (NEB/BioNordika, Finland) and 50 ng of the corresponding plasmid DNA. The conditions for the PCR reactions were the following: 30 sec initial denaturation at 98° C., followed by 28 cycles of 10 sec at 98° C., 30 sec annealing at one of the following 45/50/55/60° C., 45 sec extension at 72° C. and the final extension at 72° C. for 7 min.

Primer combination described in Table 6 produced specific DNA products having the expected sizes. The PCR products were isolated from agarose gel with GenJet Gel Extraction Kit (Thermo Fisher Scientific) according to manufacturer's instructions, digested with NruI and BamHI restriction enzymes (Thermo Fisher Scientific) and cloned into an expression vector cleaved with NruI and BamHI. Ligation mixtures were transformed into Escherichia coli XL1-Blue (AH Diagnostics) and plated on LB (Luria-Bertani) plates containing 50-100 μg/ml ampicillin. Several E. coli colonies were collected from the plates and DNA was isolated with GenJet Plasmid Miniprep Kit (Thermo Fisher Scientific). Positive clones were screened using restriction digestions. The genes encoding the Bacillus clausii man6 and Bacillus hemicellulosilyticus man7 GH5 mannanases without their own signal peptide encoding sequences were sequenced and the plasmids were named pALK4274 and pALK4273, respectively (For details see Example 6).

### Example 4. Cloning of Synthetic Bacterial Mannanase Man14

Standard molecular biology methods were used in the isolation and enzyme treatments of DNA (e.g. isolation of plasmid DNA, digestion of DNA to produce DNA fragments), in E. coli transformations, sequencing etc. The basic methods used were either as described by the enzyme, reagent or kit manufacturer or as described in the standard molecular biology handbook, e.g. Sambrook and Russell (2001). Isolation of genomic DNA was performed as described in detail by Raeder and Broda (1985).

Mannanase gene man14 from Virgibacillus soli was also cloned for Trichoderma expression. The gene encoding GH5 family mannanase Man14 from Virgibacillus soli was ordered from GenScript as a synthetic construct with codon optimization for Trichoderma reesei.

Plasmid DNA obtained from GenScript including the man14 gene was re-suspended in sterile water, digested with NruI and BamHI restriction enzymes (Thermo Fisher Scientific) according to manufacturer's instructions and cloned into an expression vector cleaved with NruI and BamHI. Ligation mixture was transformed into Escherichia coli XL1-Blue (AH Diagnostics) and plated on LB (Luria-Bertani) plates containing 50-100 μg/ml ampicillin. Several E. coli colonies were collected from the plates and DNA was isolated with GenJet Plasmid Miniprep Kit (Thermo Fisher Scientific). Positive clones were screened using restriction digestions and they were shown to contain inserts of expected sizes. Fusion sites of Virgibacillus soli man14 to the expression plasmid were sequenced and the plasmid was named pALK4414 (For details see Example 6).

### Example 5. Production of Recombinant Bacterial GH5 Mannanase Proteins in Bacillus

Expression plasmids were constructed for production of recombinant GH5 mannanase (Man6, Man7 and Man14) proteins from Bacillus clausii, Bacillus hemicellulosilyticus and Virgibacillus soli. The expression plasmids constructed are listed in Table 7. The recombinant GH5 genes (man6, man7 and man14), without their own signal sequences, were fused to the Bacillus licheniformis PaprE promoter and B. amyloliquefaciens xylanase signal peptide. The transcription termination was ensured by a strong terminator and a kanamycin resistance marker was used for selection of the transformants. The transformations were performed as described in Example 2.

TABLE 7
The expression plasmids constructed to produce Man6, Man7 and Man14
recombinant proteins from Bacillus clausii, Bacillus hemicellulosilyticus and
Virgibacillus soli in an appropriate Bacillus expression strain.

| Mannanase (GH5) protein | Expression plasmid |
|---|---|
| Man6 | pEV1 Man6 |
| Man7 | pEV1 Man7 |
| Man14 | pEV1 Man14 |

The GH5 production of the transformants was analyzed from the culture supernatants of the shake flask cultivations. The transformants were inoculated from the LB plates to shake flasks containing 2% glucose, 6% corn steep powder, 1.3% (NH4)2HPO4, 0.05% MgSO4×7H2O and 0.5% CaCl2). pH was adjusted to pH 7.5. The GH5 protein production of the transformants was analyzed from culture supernatants after growing them for 30 hours at 37° C., 180 rpm. Heterologous production of recombinant proteins was analyzed by SDS-PAGE with subsequent Coomassie staining.

The best producing transformants were chosen to be cultivated in laboratory scale bioreactors. The transformants were cultivated in bioreactors at 37° C. under protein inducing conditions and additional feeding until a suitable yield was reached. The supernatants were recovered for application tests by centrifugation or filtration.

### Example 6. Production of Recombinant Bacterial GH5 Mannanase Proteins in Trichoderma reesei

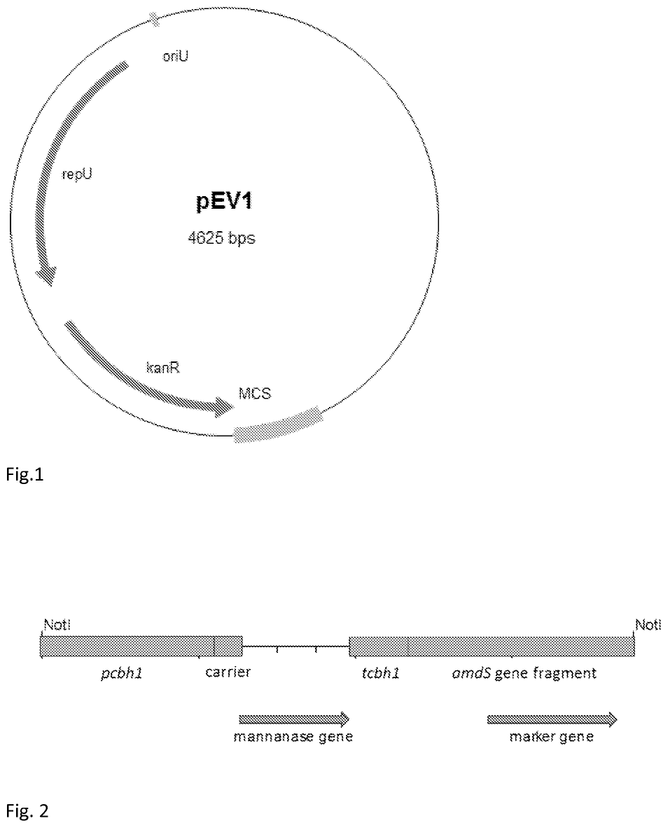

Expression plasmids were constructed for production of recombinant GH5 mannanase (Man6, Man7 and Man14) proteins from Bacillus clausii, Bacillus hemicellulosilyticus and Virgibacillus soli (See Examples 3 and 4) in Trichoderma reesei. The expression plasmids constructed are listed in Table 8. The recombinant GH5 genes (man6, man7 and man14), without their own signal sequences, were fused to the T. reesei cel7A/cbh1 promoter with T. reesei cel6A/cbh2 CBM carrier and linker followed by Kex2 protease recognition site. The transcription termination was ensured by the T. reesei cel7A/cbh1 terminator and the A. nidulans amdS marker gene was used for selection of the transformants as described in Paloheimo et al. (2003). The linear expression cassettes (FIG. 2) were isolated from the vector backbones after NotI digestions and were transformed into T. reesei protoplasts. The host strains used does not produce any of the four major T. reesei cellulases (CBHI, CBHII, EGI, EGII). The transformations were performed as in Penttilä et al. (1987) with the modifications described in Karhunen et al. (1993), selecting acetamidase as a sole nitrogen source (amdS marker gene). The transformants were purified on selection plates through single conidia prior to sporulating them on PD.

TABLE 8
The expression cassettes constructed to produce Man6, Man7 and Man14
recombinant proteins from Bacillus clausii, Bacillus hemicellulosilyticus
and VirgibaciHus soli in Trichoderma reesei.The overall structure
of the expression cassettes was as described in FIG. 2.

| Mannanase (GH5) | | |
|---|---|---|
| protein | Expression plasmid | Expression cassette ^{(a} |
| Man6 | pALK4274 | 7.0 kb NotI |
| Man7 | pALK4273 | 7.5 kb NotI |
| Man14 | pALK4414 | 7.6 kb NotI |

^{(a} The expression cassette for T. reesei transformation was isolated from vector backbone by using NotI digestion.

The mannanase production of the transformants was analyzed from the culture supernatants of the shake flask cultivations. The transformants were inoculated from the PD slants to shake flasks containing 50 ml of complex lactose-based cellulase inducing medium (Joutsjoki at al. 1993) buffered with 5% KH₂PO₄. The GH5 protein production of the transformants was analyzed from culture supernatants after growing them for 7 days at 30° C., 250 rpm. Heterologous production of recombinant proteins was analyzed by SDS-PAGE with subsequent Coomassie staining.

The best producing transformants were chosen to be cultivated in laboratory scale bioreactors. The transformants were cultivated in bioreactors either on batch or by additional feeding type of process under protein inducing conditions at a typical mesophilic fungal cultivation temperature and slightly acidic conditions. The cultivation was continued until depletion of the medium sugars or until suitable yield was reached. The supernatants were recovered for application tests by centrifugation or by filtration.

### Example 7. Assay of Galactomannanase Activity by DNS-Method

Mannanase activity (MNU) was measured as the release of reducing sugars from galactomannan (0.3 w/w-%) at 50° C. and pH 7.0 in 5 min. The amount of released reducing carbohydrates was determined spectrophotometrically using dinitrosalicylic acid.

Substrate (0.3 w/w-%) used in the assay was prepared as follows: 0.6 g of locust bean gum (Sigma G-0753) was in 50 mM sodium citrate buffer pH 7 (or citrate phosphate buffer pH 7) at about 80° C. using a heating magnetic stirrer and heated up to boiling point. The solution was cooled and let to dissolve overnight in a cold room (2-8° C.) with continuous stirring and insoluble residues were removed by centrifugation. After that solution was filled up to 200 ml by buffer. Substrate was stored as frozen and melted by heating in a boiling water bath to about 80° C., cooled to room temperature and mixed carefully before use.

DNS reagent used in the assay was prepared by dissolving 50 g of 3.5-dinitrosalisylic acid (Sigma D-550) in about 4 liter of water. With continuous magnetic stirring 80.0 g of NaOH was gradually added and let to dissolve. An amount of 1500 g of Rochelle Salt (K-Na-tartrate, Merck 8087) was added in small portions with continuous stirring. The solution that was cautiously warmed to a maximum temperature of 45° C., was cooled to room temperature and filled up to 5000 ml. After that it was filtered through Whatman 1 filter paper and stored in a dark bottle at room temperature.

The reaction was first started by adding 1.8 ml of substrate solution to each of the two test tubes and let to equilibrate at 50° C. for 5 minutes, after which 200 μl of suitably diluted enzyme solution was added to one of the tubes, mixed well with vortex mixer and incubated exactly for 5 min at 50° C. Enzyme blanks didn't need to be equilibrated or incubated. The reaction was stopped by adding 3.0 ml of DNS reagent into both tubes and mixed. 200 μl of sample solution was added to the enzyme blank tubes. Both tubes were placed in a boiling water bath. After boiling for exactly 5 minutes, the tubes were placed in a cooling water bath and allow them to cool to room temperature. The absorbance of sample was measured against the enzyme blank at 540 nm and activity was read from the calibration curve and multiplied by the dilution factor. A suitable diluted sample yielded an absorbance difference of 0.15-0.4.

Standard curve was prepared 20 mM from mannose stock solution by dissolving 360 mg of mannose (SigmaM-6020, stored in a desiccator) in assay buffer and diluted to solutions containing 3, 6, 10 and 14 μmol/ml of mannose. Standards were handled like the samples except for incubating at 50° C. The absorbances were measured against the reagent blank (containing buffer instead of standard dilution of mannose) at 540 nm. Calibration curve was constructed for every series of assays.

One mannanase unit (MNU) was defined as the amount of enzyme that produces reductive carbohydrates having a reductive power corresponding to one nmol of mannose from galactomannan in one second under the assay conditions (1 MNU=1 nkat).

### Example 8. Purification of Man6 Mannanase

Cells and solids were removed from the fermentation culture medium by centrifugation for 10 min, 4000 g at 4° C. The supernatant of 10 ml was used for protein purification. The sample was filtered through 0.44 μm PVDF membrane (Millex-HV, Merck Millipore Ltd, Carrigtwohill, IRL). The filtrate was loaded onto a HiPrep 26/10 Desalting column (GE Healthcare, Uppsala, Sweden) equilibrated in 20 mM HEPES pH 7. The desalted sample was then loaded onto a 5 ml HiTrap Q HP column (GE Healthcare, Uppsala, Sweden) pre-equilibrated with 20 mM HEPES pH 7. After sample loading, the column was washed with the same buffer for 20 ml. Proteins were eluted with linear salt gradient 20 mM HEPES, 500 mM NaCl pH 7 in 15 CVs. Fractions of 5 ml were collected and analyzed on SDS-PAGE. The fractions containing target protein were combined and concentrated to 2 ml using Vivaspin 20, 10 kDa MWCO ultrafiltration devices (GE Healthcare). The concentrated sample was further fractionated using Superdex 75 26/60 gel-filtration column equilibrated with 20 mM MES, 200 mM NaCl pH 6.5. Fractions of 2 ml were collected and analyzed by SDS-PAGE. Fractions containing pure mannanase were combined. Other mannanases were purified using the same protocol but changing the buffer composition in desalting and ion exchange steps. Buffer compositions are shown in Table 9.

TABLE 9
Buffers used in ion exchange chromatography

| | Buffers used in ion |
|---|---|
| Mannanase | exchange chromatography |
| Man6 | 20 mM HEPES pH 7 |
| Man7 | 20 mM HEPES pH 7 |
| Man14 | 20 mM MES pH 6 |

Purified samples were above 95% pure.

Enzyme content of the purified sample was determined using UV absorbance 280 nm measurements. Excitation coefficients for each mannanases were calculated on the bases of amino acid sequence of the enzyme by using ExPASy (Server http://web.expasy.org/protparam/). (Gasteiger et al. 2005).

The enzyme activity (MNU) of purified samples was measured as release of reducing sugars as described in Example 7.

The specific activity (MNU/mg) of mannanases was calculated by dividing MNU activity of purified sample with the amount of purified enzyme. Obtained values were used for calculating enzyme dosages used in Examples 10 and 11.

### pH Profiles of Mannanases

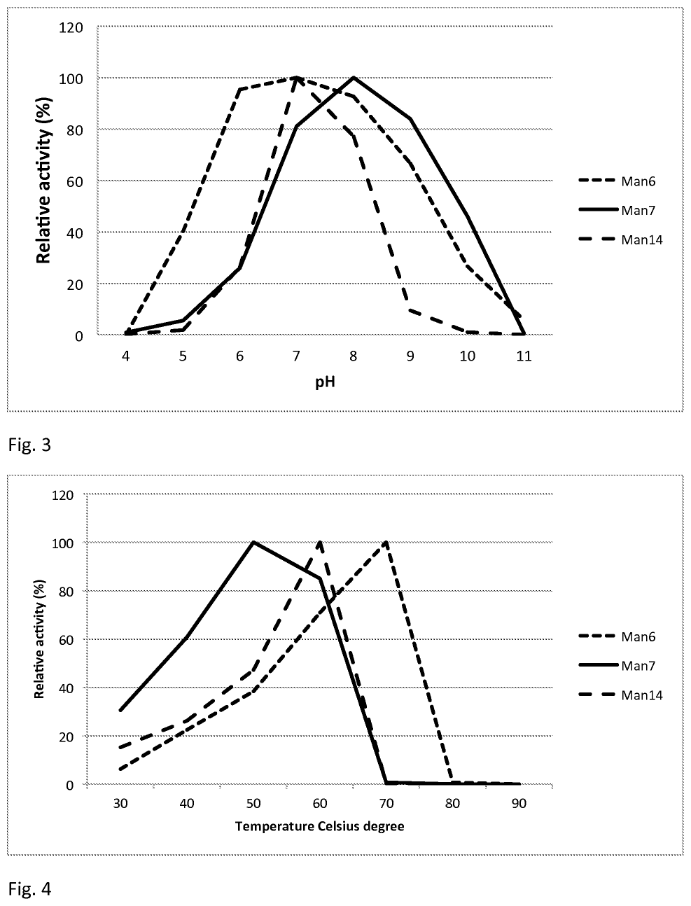

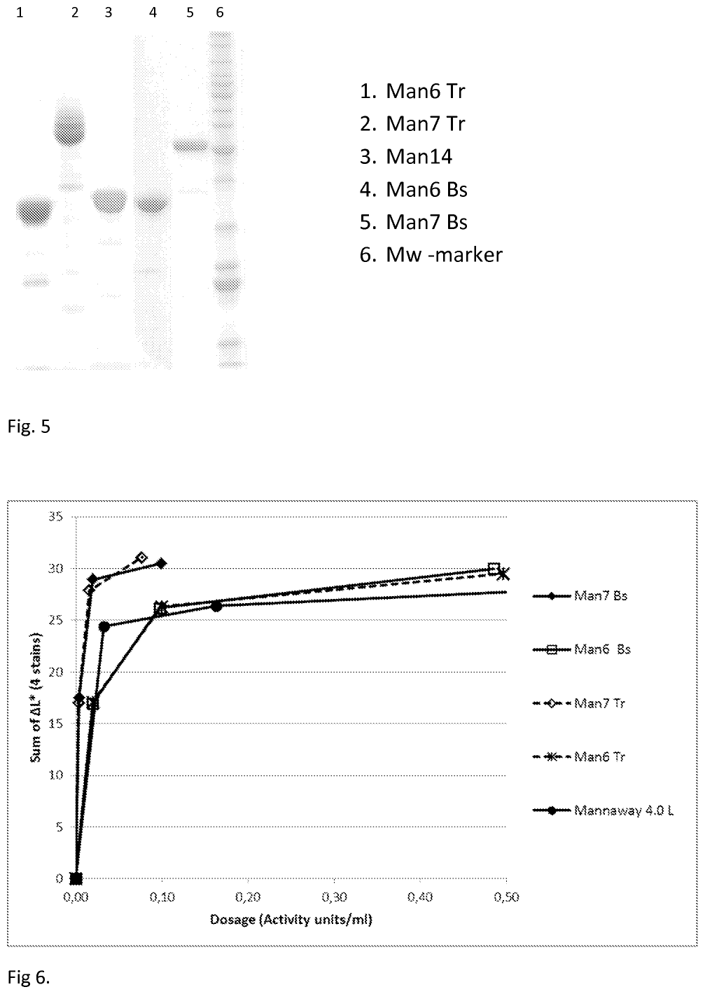

The pH profiles of purified mannanases were determined using the beta-mannazyme tablet assay Azurine-crosslinked carob galactomannan (T-MNZ 11/14) from Megazyme with minor modifications to the suggested protocol. The linearity of the assay has been checked with each purified enzymes. The assay was performed in 40 mM Britton-Robinson buffer adjusted to pH values between 4 and 11. The enzyme solution was diluted into the assay buffer and 500 μl of enzyme solution was equilibrated at 50° C. water bath for 5 min before adding one substrate tablet. After 10 minutes, the reaction was stopped by adding 10 ml 2% Tris pH 12. The reaction tubes were left at room temperature for 5 min, stirred and the liquid filtered through a Whatman No. 1 paper filter. Release of blue dye from the substrate was quantified by measuring the absorbance at 595 nm. Enzyme activity at each pH was reported as relative activity where the activity at the pH optimum was set to 100%. The pH profiles were shown in FIG. 3.

Relative activity (%) of mannanase is calculated by dividing mannanase activity of a sample by the mannanase activity of a reference sample. In the case of pH profile, the reference sample is a sample at the optimal pH. In the case of temperature profile the reference sample is a sample at the optimal temperature.

### Temperature Profiles of Mannanases

The temperature optimum of purified mannanases was determined using the beta-mannazyme tablet assay Azurine-crosslinked carob galactomannan (T-MNZ 11/14) from Megazyme with minor modifications to suggested protocol. The assay was performed at temperatures varying between 30-90° C. for 10 minutes in 40 mM Britton-Robinson buffer pH7. Enzyme activity was reported as relative activity where the activity at temperature optimum was set to 100%. The temperature profiles were shown in FIG. 4.

### Temperature and pH Characteristics of Mannanases

Man6 has a molecular mass between 30-35 kDa. The optimal temperature of the enzyme at pH 7 is from 50° C. to 70° C. Said enzyme has pH optimum at the pH range of at least pH 6 to pH 9 at 50° C. The optimal temperature and pH optimum were determined using 10 min reaction time and Azurine-crosslinked carob galactomannan as a substrate.

Man7 has a molecular mass between 50-55 kDa. The optimal temperature of the enzyme at pH 7 is from 40° C. to 60° C. Said enzyme has pH optimum at the pH range of at least pH 7 to pH 10 at 50° C. The optimal temperature and pH optimum were determined using 10 min reaction time and Azurine-crosslinked carob galactomannan as a substrate.

Man14 has a molecular mass between 30-40 kDa. The optimal temperature of the enzyme at pH 7 is from 50° C. to 60° C. Said enzyme has pH optimum at the pH range of at least pH 7 to pH 8 at 50° C. The optimal temperature and pH optimum were determined using 10 min reaction time and Azurine-crosslinked carob galactomannan as a substrate.

### Example 9. Stain Removal Performance of Man6 and Man7 Mannanases with Commercial Detergents without Bleaching Agents

Man6 and Man7 mannanases produced in Bacillus (as described in Example 5) and in Trichoderma (as described in Example 6), were tested for their ability to remove mannanase sensitive standard stains at 40° C. and water hardness of 16° dH with commercial detergents without bleaching agents and compared to commercial mannanase preparation Mannaway® 4.0 L (Novozymes). The following artificially soiled test cloths from Center for test material B.V. (the Netherlands) were used: Chocolate pudding mannanase sensitive on cotton (E-165), Locust bean gum, with pigment on cotton (C-S-73) and on polyester/cotton (PC-S-73) and Guar gum with carbon black on cotton (C-S-43). The fabric was cut in 6 cm×6 cm swatches and 2 pieces of each were used in test.

Commercial heavyduty liquid detergent A containing all other enzymes except mannanase was used at concentration of 4.4 g per liter of wash liquor and Commercial Color detergent powder without enzymes was used at 3.8 g/l. Detergent containing wash liquors we prepared in synthetic tap water with hardness of 16° dH. Protease Savinase® 16 L (0.5 w/w %) and amylase Stainzyme® 12 L (0.4 w/w %) was added into hard water used with commercial Color detergent powder, the liquid detergent already contained amylase and protease. pH of the wash liquor of Color detergent powder was approximately 10 and with the liquid detergent approximately 8.3.

Mannanase dosages were in range 0-0.2/0.25% of detergent weight but for the evaluation the dosages were calculated as enzyme activity units (MNU) per ml wash liquor or as mg of active enzyme protein (AEP) per l of wash liquor. Activity was measured as described in Example 7. AEP content of each preparation was calculated by dividing the enzyme activity with specific activity, defined in Example 8. Control sample contained the detergent solution but no mannanase.

For synthetic tap water with hardness of 16° dH the following stock solutions were prepared in deionized water (Milli-Q or equivalent):

Stock solution with 1000° d Calcium-hardness: CaCl₂)×2 H2O (1.02382.1000, Merck KGaA, Germany) 26.22 g/l

Stock solution with 200° d Magnesium-hardness: MgSO4×7 H2O (1.05886.1000, Merck KGaA, Germany) 8.79 g/l H2O

NaHCO3 stock solution: NaHCO3 (1.06329.1000 Merck KGaA, Germany) 29.6 g/l

13.3 ml CaCl₂) solution, 13.3 ml MgSO4 solution and 10.0 ml of freshly made NaHCO3 solution were added in volumetric flask in the given order, made up to 1 liter with deionized water and mixed. The hardness of water was determined by complexometric titration and found correct.

Stain removal treatments were performed in Atlas LP-2 Launder-Ometer as follows. Launder-Ometer was first preheated to 40° C. Then detergent, 250 ml synthetic tap water with hardness of 16° dH and diluted enzyme (<1.0 ml) were added into 1.2 liter containers. Stains were added and the Launder-Ometer was run at 40° C. for 60 min with a rotation speed of 42 rpm. After that the swatches were carefully rinsed under running water and dried overnight at indoor air, on a grid protected against daylight.

The stain removal effect was evaluated by measuring the colour as reflectance values with Konica Minolta CM-3610A spectrophotometer using L*a*b* color space coordinates (illuminant D65/10°, 420 nm cut). Fading of the stains, indicating mannanase performance (stain removal efficiency) was calculated as ΔL* (delta L*), which means lightness value L* of enzyme treated fabric minus lightness value L* of fabric treated with washing liquor without mannanase (control). Final results (total stain removal effect) were shown as sum of ΔL* of each stains. Color values of each stains were average of 2 swatches.

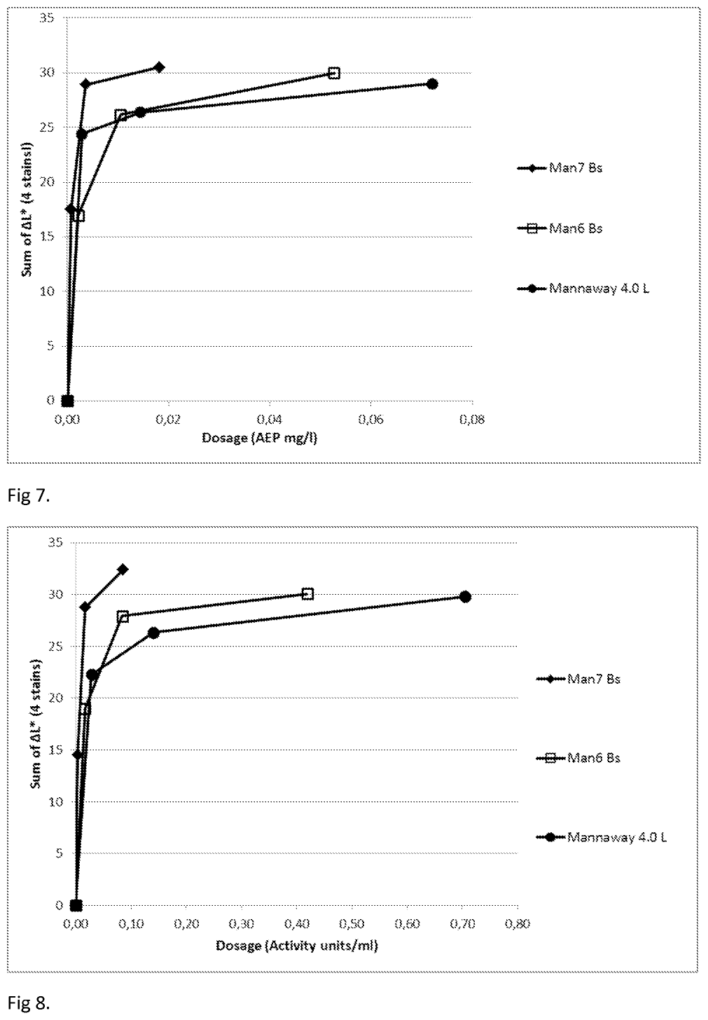

The results obtained with commercial liquid detergent are shown in FIGS. 6-7. The mannanases according to the aspects of the disclosed embodiments have similar (Man6) or considerably better (Man7) stain removal performance with liquid detergent when dosed as activity units or as active enzyme protein compared to commercial mannanase preparation Mannaway® 4.0 L. Similar performance was obtained with Man6 and Man7 regardless of the expression host, Bacillus or Trichoderma (FIG. 6).

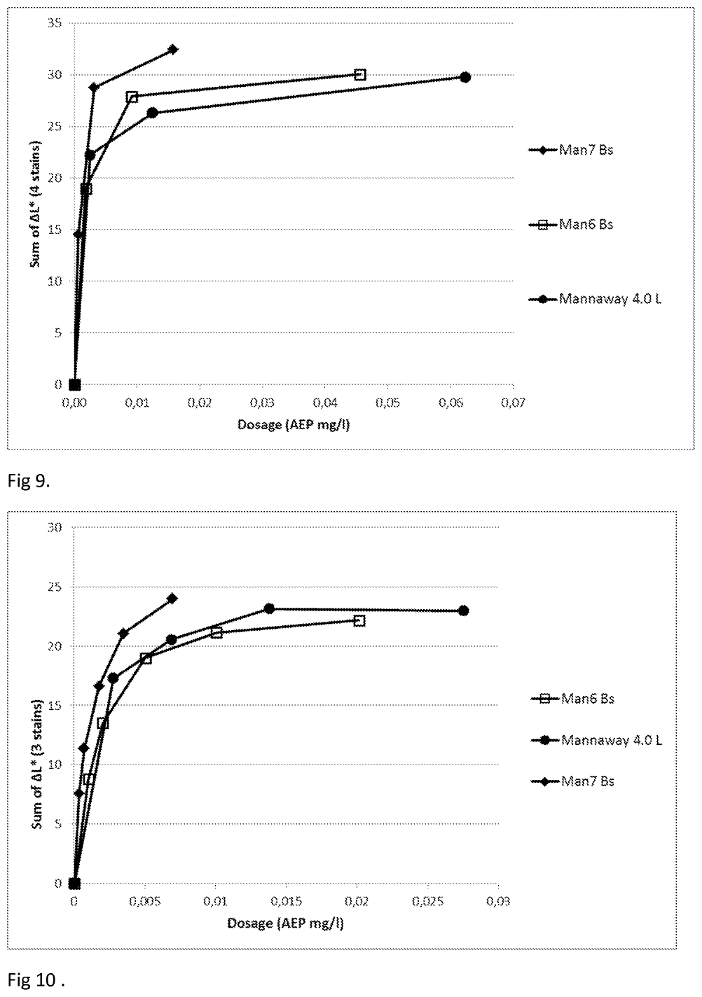

The results obtained with commercial color detergent powder (FIGS. 8-9) show that the mannanases according to the aspects of the disclosed embodiments have better stain removal performance with color detergent powder when dosed as activity units or as active enzyme protein compared to commercial mannanase preparation Mannaway® 4.0 L.

### Example 10. Stain Removal Performance Man6 and Man7 Mannanases with Bleach Containing Detergent

Man6 and Man7 mannanases produced in Bacillus (as described in Example 5) were tested for their ability to remove mannanase sensitive standard stains at 40° C. and water hardness of 16° dH with commercial bleach detergent powder and compared to commercial mannanase preparation Mannaway® 4.0 L (Novozymes). Test system was similar to described in Example 9, except three different artificially soiled test cloths from Center for test material B.V. (the Netherlands) were used: Chocolate pudding mannanase sensitive on cotton (E-165), Locust bean gum, with pigment on cotton (C-S-73) and Guar gum with carbon black on cotton (C-S-43). Commercial Color detergent powder was used at concentration of 4.2 g per liter of wash liquor and pH of the wash liquor was approx. 9.5. Protease Savinase® 16 L (0.5 w/w %) and amylase Stainzyme® 12 L (0.4 w/w %) were added into hard water used in test, since the detergent didn't contain any enzymes.

The color of the swatches after treatment was measured and results calculated as sum of ΔL* of each 3 stains as described in Example 9.

The results (FIG. 10) obtained with commercial bleach containing detergent indicate that the mannanase according to the aspects of the disclosed embodiments (Man7) has considerably better stain removal performance compared to commercial mannanase Mannaway® 4.0 L when dosed as active enzyme protein. With Man6 at least similar performance compared to a commercial bacterial mannanase is obtained.

### Example 11. Stain Removal Performance Man14 Mannanase with Commercial Liquid Detergent

Man14 mannanase produced in Bacillus (as described in Example 5) was tested for their ability to remove mannanase sensitive standard stains at 40° C. and water hardness of 16° dH with commercial heavy duty liquid detergent B and compared to commercial mannanase preparation Mannaway® 4.0 L (Novozymes). Test system was similar to that described in Example 9, except two different artificially soiled test cloths from Center for test material B.V. (the Netherlands) were used: Chocolate pudding mannanase sensitive on cotton (E-165) and Locust bean gum, with pigment on cotton (C-S-73). Commercial heavy duty liquid detergent B was used at concentration of 5 g per liter of wash liquor and pH of the wash liquor was approximately 8.3. Protease Savinase® 16 L (0.5 w/w %) and amylase Stainzyme® 12 L (0.4 w/w %) were added into hard water used in test, since the detergent didn't contain any enzymes.

The color of the swatches after treatment was measured and results calculated as sum of ΔL* of each 2 stains as described in Example 9.

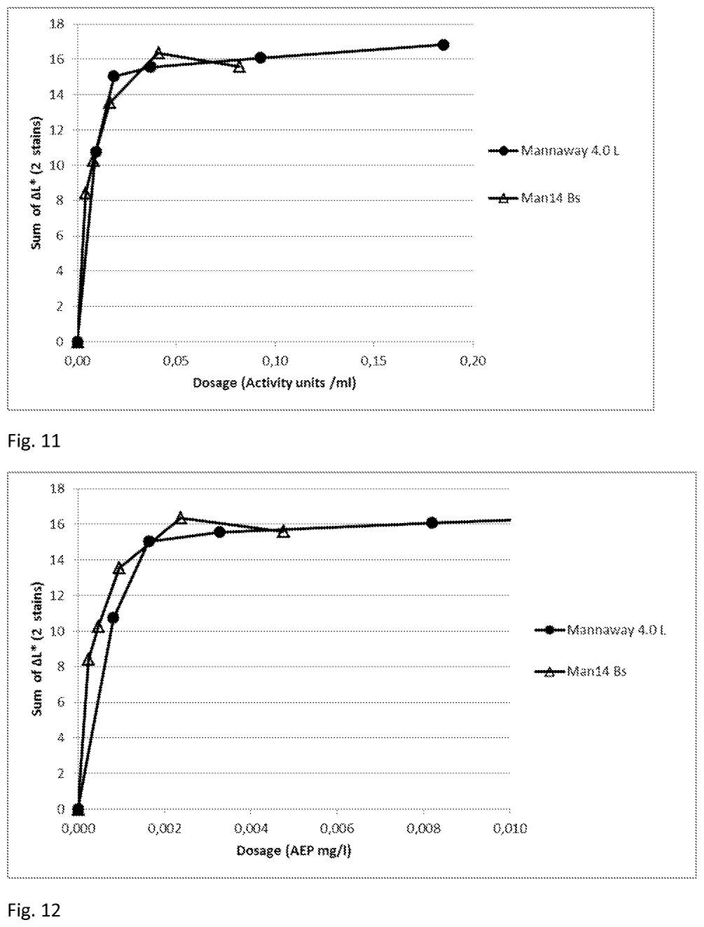

The results (FIGS. 11-12) obtained with commercial liquid containing detergent indicate Man14 had good performance in a liquid detergent, comparable to commercial product, when dosed either as activity units or as active enzyme protein.

### Example 12. Stability of Man6 and Man7 Mannanases in Commercial Liquid Detergents

The stability of Man6 and Man7 mannanase preparations produced in Bacillus were tested in OMO Color liquid obtained from local super market and compared to commercial mannanase preparation Mannaway® 4.0 L. Mannanase preparations were added 0.5% w/w-% in detergents and samples were incubated in plastic tubes with caps at 37° C. for 5 weeks. The activity was measured at certain intervals by activity assay described in Example 7 except using 30 min incubation time. Results were calculated as residual activity (%), which was obtained by dividing the activity of a sample taken at certain time point by initial activity of the sample.

The stability of Man7 produced both in Bacillus and Trichoderma and Man6 produced in Trichoderma were tested against Mannaway® 4.0 L also in commercial liquid heavyduty detergent A containing protease but no mannanase. In this test 1%-(w/w) of mannanases were used and samples incubated for 37° C. for 12 weeks.

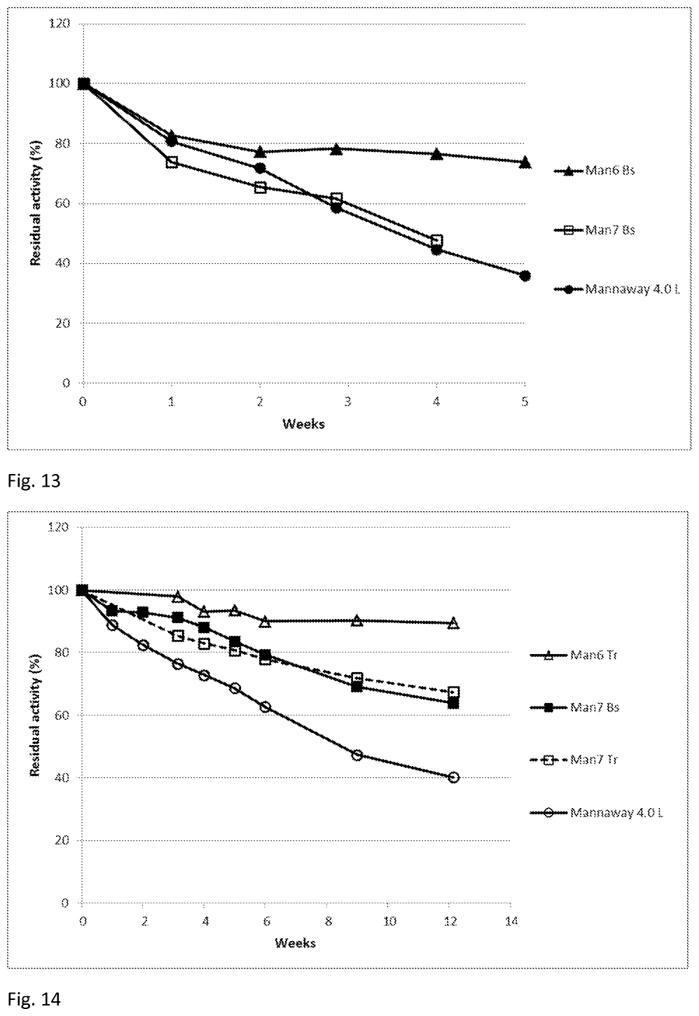

The results in Omo Color (FIG. 13) show that Man6 had considerably better and Man7 similar stability compared to Mannaway® 4.0 L. Both Man7 and especially Man6 were more stable than Mannaway® 4.0 L with another commercial liquid detergent A, as shown in FIG. 14. Results obtained in another test at same conditions showed that Man6 had similar stability regardless of the expression host, Bacillus or Trichoderma (data not shown).

The results of the stability experiments show that the mannanase according to the present disclosure is stabile in detergents for several weeks even when stored at high temperature like 37° C. The stability of the mannanases according to the present disclosure (Man6 and Man7) is improved compared to a commercial bacterial mannanase in liquid detergent.

### Example 13. Efficiency Study with Mannanase Alone and in Combination with a Non-Starch Polysaccharide (NSP) Degrading Enzyme in Broilers

Effects of recombinant mannanases of the present disclosure are studied on growth in broilers. Ultrafiltrate of the fermentation broth including the recombinant mannanase is dried and target levels applied to a pelleted broiler diet alone or in combination with a commercial available xylanase based product.

A control diet based on corn and dehulled sol-vent extracted soybean meal is fed without enzyme or added by different levels of the recombinant mannanase of the present disclosure alone or in combination with a standard dose of a commercial xylanase.

Initial weight of the broilers is between 30 g and 50 g. The trial lasts between three and five weeks. Each treatment consists at minimum of six replicates with 10 broilers each. In each case the diet is analysed for moisture, crude protein, crude fibre, fat, ash, and enzyme protein.

Five diets are prepared:

1) unsupplemented control (BD)

2) BD+mannanase 1—500 mg/kg

3) BD+mannanase 1—1000 mg/kg

4) BD+mannanase 1≤500 mg/kg+xylanase 1-10 mg/kg

5) BD+xylanase 1-10 mg/kg

Health status and mortality of the animals is checked daily by visual inspection. At days 0, 14, and 35 body weight gain (BW), feed intake (FI), and feed-conversion ratio (FCR) are measured. FCR is calculated as the total feed consumed divided by the weight gain during the same period. Determination of the effect of the recombinant mannanases is based on the comparison to those animals fed the same diet or the same diet but added by xylanase.

### Example 14. Instant Coffee Production

Pure mannan is the main storage polysaccharide component of coffee endosperms and is responsible for their high viscosity, which negatively affects the technological processing of instant coffee and increases energy consumption during drying. Those effects are attributed to mannan forming hard, insoluble crystalline structures. β-mannanase, often together with other enzymes such as pectinase and cellulase, is added during the concentration step of instant coffee production to reduce viscosity in coffee extracts. Mannanase is also be employed for hydrolyzing galactomannans present in a liquid coffee extract in order to inhibit gel formation during freeze drying of instant coffee. Furthermore, due to the use of enzymatic treatment the coffee bean extracts can be concentrated by a low cost procedure such as evaporation.

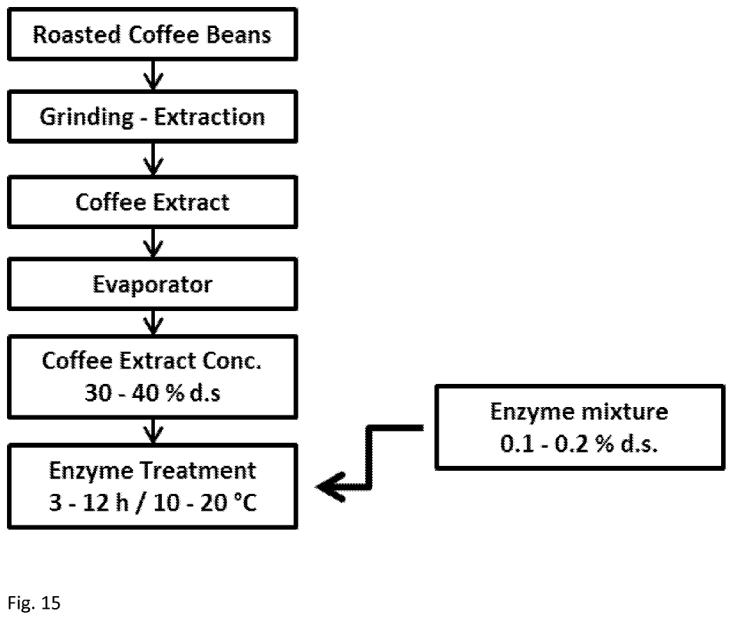

The test is performed according the following flow-chart of FIG. 15 at temperatures of 10° C. and a enzyme dosage of 0.15% d.s.

Mannanases of the present disclosure are tested in mixture composed of different enzymes, such as pectinases and cellulases.

The viscosity of the coffee extract increases significantly over time under standard process conditions. However, the viscosity is significantly reduced using the enzyme mixture containing the mannanases of the present disclosure resulting an improved downstream processing such as spray- or freeze drying.

### Example 15. Pineapple Processing

In particular, mannanase is useful for pineapple mill juice extraction and clarification, as pineapple contains a large fraction of mannans, including glucomannans and galactomannans.

Mannanase helps to improve extraction of valuable fruit components, lower the viscosity of fruit juice prior to concentration, and increase filtration rate and stability of the final product.

The pineapples are crushed in a meat grinder and fill 500 g mash in a 1000 ml beaker. The enzyme is applied at 21° C. with a reaction time of 60 minutes. The mash is then pressed with a small Hafico press according to the press protocol: 0 bar 2 min-50 bar 2 min-100 bar 2 min-150 bar 2 min-200 bar 1 min-300 bar 1 min-400 bar 1 min. The obtained juice is then centrifuged at 4500 rpm for 5 minutes and analyzed for turbidity and viscosity.

Mannanases of the present disclosure are tested in enzyme mixtures A, B and C (Table 10).

The enzymes are first diluted with tab water before being added to the pineapple mash

TABLE 10
Enzyme mixtures

| | Enzyme | Dosage | 5 ml of |
|---|---|---|---|
| | activity | [ppm] | [% enzyme solution] |
| blank | | | 5 ml H2O |
| Mixture A | Pectinase | 50 | 0.50% |
| Mixture B | Pectinase + | 50 | 0.50% |
| | Arabanase | | |
| Mixture C | Pectinase + | 50 | 0.50% |
| | Mannanase | | |

Applying mannanases of the aspects of the disclosed embodiments leads to increased yield and lower turbidity of juice in pineapple processing.

### Example 16. Mannanase Treatment of Soya Beans for Soya Milk Production

For the enzymatic treatment of soya beans to get soya milk the “hot process” is commonly used. For the hot soya milk process the dried soya beans were mixed and crushed in a mixer with boiling tap water in a ratio of 1:7 (soaked beans: water). The whole soya slurry is cooled down to 50-55° C. before enzyme addition. The pH level for the soya slurry should be around pH 6.5 and can be adjusted with NaHCO₃. The mannanase enzyme is dosed at 1 kg/t of dried soya beans into the slurry and stirred for 30 min. After completion of the reaction time, the slurry is pressed using a laboratory press to obtain the final product: soya milk. In order to ensure the same pressing profile, the pressure as well as the corresponding pressing time is specified, as shown in table 11. Besides the sample for enzymatic reaction, a control sample without any enzyme is prepared, in which the enzyme solution was replaced with water.

TABLE 11
Press scheme

| pressure [bar] | 0 | 50 | 100 | 300 |
|---|---|---|---|---|
| time [min] | 2 | 2 | 2 | 1 |

After pressing the soya milk is heated in a microwave until boiling to stop the enzyme reaction. Analysis of the soya milk:

Yield in gram/time

° Brix, which gives a direct correlation of the amount of sugar in the soy milk, is determined with a refractometer

The turbidity of the juice is measured with a NTU-photometer, which measures the nephelometric turbidity.

The brightness will be measured with a LAB-measurement

Protein content is determined with a CN-Analyser (combustion method)

Flavour

Soya milk treated with the mannanases of the aspects of the disclosed embodiments show a increased yield, brighter colour, increased ° Brix, a lower turbidity, a higher protein content and a better taste (off flavour removal).

### Example 17. Wash Performance of Liquid Detergent Compositions According to the Present Disclosure

The wash performance of liquid detergent compositions according to present disclosure was determined by using standardized stains obtainable from CFT (Center for Testmaterials) B.V., Vlaardingen, Netherlands (“OFT”), Eidgenössische Material- and Prüfanstalt Testmaterialien AG [Federal materials and testing agency, Testmaterials], St. Gallen, Switzerland (“EMPA”) and Warwick Equest Ltd Unit 55, Consett Business Park, Consett, County Durham (“Equest”).

A liquid washing agent with the following composition was used as base formulation (all values in weight percent):

TABLE 12

| | | Active substance |
|---|---|---|
| | Active substance | detergent |
| Chemical name | raw material [%] | formulation [%] |
| Water demin. | 100 | Rest |
| Alkyl benzene sulfonic acid | 96 | 2-7 |
| Anionic surfactants | 70 | 6-10 |
| C12-C18 Fatty acid sodium | 30 | 1-4 |
| salt | | |
| Nonionic surfactants | 100 | 4-7 |
| Phosphonates | 40 | 0.1-2 |
| Citric acid | 100 | 1-3 |
| NaOH | 50 | 1-4 |
| Boronic acid | 100 | 0.1-2 |
| Antifoaming agent | 100 | 0.01-1 |
| Glycerol | 100 | 1-3 |
| Enzymes | 100 | 0.1-2 |
| Preserving agent | 100 | 0.05-1 |
| Ethanol | 93 | 0.5-2 |
| Optical brightener | 90 | 0.01-1 |
| Perfume | 100 | 0.1-1 |
| Dye | 100 | 0.001-0.1 |

The pH of the detergent composition was between 8.2-8.6.

Based on this base formulation, liquid detergent compositions 1 and 2 were prepared by adding respective enzymes as indicated below:

Composition 1: Enzyme according to SEQ ID NO:12 (Man6)

Composition 2: Enzyme according to SEQ ID NO:16 (Man7)

The wash was performed as follows according to the AISE Method: 3.5 kg Clean ballast cloth, 4 SBL Cloths, Miele washing machine, 20° C. and 40° C. Short program.

All mannanases were added in the same amounts based on total protein content.

The dosing ratio of the liquid washing agent was 4.0 grams per liter of washing liquor. The washing procedure was performed for 60 minutes at a temperature of 20° C. and 40° C., the water having a water hardness between 15.5 and 16.5° (German degrees of hardness).

The results obtained are the difference values between the remission units obtained with the detergents and the remission units obtained with the detergent containing the commercially available reference mannanase (Mannaway 4.0 L, obtained from Novozymes). A positive value therefore indicates an improved wash performance of the detergent compositions comprising the mannanases of present disclosure compared to the same detergent composition comprising the reference mannanase. Within the washing test a large range of stains were tested.

The whiteness, i.e. the brightening of the stains, was determined photometrically as an indication of wash performance. A Minolta CM508d spectrometer device was used, which was calibrated beforehand using a white standard provided with the unit.

The results obtained are the difference values between the remission units obtained with the detergents and the remission units obtained with the detergent containing the enzyme combinations. A positive value therefore indicates an improved wash performance due to the enzyme combinations present in the detergent. Mannanases of the disclosure in detergent compositions show improved performance on a variety of mannan comprising stains.

TABLE 13

| | 20° C. | | 40° C. | |
|---|---|---|---|---|
| | Comp. | Comp. | Comp. | Comp. |
| Stain | 1 | 2 | 1 | 2 |
| Chocolate Ice Cream | 1.3 | 4.2 | n.d. | n.d. |
| (Equest) | | | | |
| Carte Dór Chocolate Ice Cream | n.d. | 3.3 | n.d. | 0.7 |
| (Equest) | | | | |
| Cocoa [CO] (Equest) | n.d. | 2.3 | n.d. | n.d. |
| Mayonnaise/Carbon black color | 1.3 | 4.3 | 1.1 | 2.2 |
| (CFT CS05S [CO]) | | | | |
| Salad dressing, with natural black | 1.2 | 3.5 | 1.2 | 2.6 |
| (CFT CS06 [CO]) | | | | |
| Lipstick, diluted, Red | n.d. | 1.5 | n.d. | 0.7 |
| (CFT CS216 [CO]) | | | | |

### Example 18. Wash Performance of Powder Detergent Compositions According to the Present Disclosure

The wash performance of powder detergent compositions according to present disclosure was determined by using standardized stains obtainable from CFT (Center for Testmaterials) B.V., Vlaardingen, Netherlands (“CFT”), Eidgenössische Material- and Prüfanstalt Testmaterialien AG [Federal materials and testing agency, Testmaterials], St. Gallen, Switzerland (“EMPA”) and Warwick Equest Ltd Unit 55, Consett Business Park, Consett, County Durham (“Equest”).

A solid washing agent with the following composition was used as base formulation (all values in weight percent):

TABLE 14

| | Active | Active |
|---|---|---|
| Chemical | substance raw | substance detergent |
| name | material [%] | formulation [%] |
| Water demin. | 100 | 1-4 |
| Alkyl benzene sulfonic acid | 97 | 9-13 |
| Nonionic surfactants | 100 | 4-7 |
| Percarbonates | 88 | 9-13 |
| TAED | 92 | 1-5 |
| Phosphonates | 60 | 0.1-3 |
| Polyacrylates | 45 | 1-4 |
| Sodium silicate | 40 | 5-10 |
| Sodium carbonate | 100 | 18-22 |
| Carboxymethylcellulose | 69 | 1-4 |
| Soil release polymer | 100 | 0.1-1 |
| Optical brightener | 70 | 0.1-1 |
| Antifoaming agent | t.q. | 0.01-1 |
| Sodium sulfate | 100 | 22-30 |
| Enzymes | 100 | 0.1-1 |
| Perfume | 100 | 0.1-1 |
| NaOH | 100 | 0.1-1 |
| Rest | — | 1-4 |

Based on this base formulation, solid detergent compositions 3 and 4 were prepared by adding respective enzymes as indicated below:

Composition 3: Enzyme according to SEQ ID NO:12 (Man6)

Composition 4: Enzyme according to SEQ ID NO:16 (Man7)

The wash was performed as follows according to the AISE Method: 3.5 kg Clean ballast cloth, 4 SBL Cloths, Miele washing machine, 20° C. and 40° C. Short program. All mannanases were added in the same amounts based on total protein content.

The dosing ratio of the powder washing agent was 3.8 grams per liter of washing liquor. The composition of the detergent is listed in Table 14. The washing procedure was performed for 60 minutes at a temperature of 20° C. and 40° C., the water having a water hardness between 15.5 and 16.5° (German degrees of hardness).

The whiteness, i.e. the brightening of the stains, was determined photometrically as an indication of wash performance. A Minolta CM508d spectrometer device was used, which was calibrated beforehand using a white standard provided with the unit.

The results obtained are the difference values between the remission units obtained with the detergents and the remission units obtained with the detergent containing the reference mannanase (Mannaway 4.0 L, obtained from Novozymes). A positive value therefore indicates an improved wash performance of the mannanases in the detergent. Mannanases of the present disclosure show improved performance on several stains in Table 15. Therefore, it is evident that mannanases according to the present disclosure show improved wash performance compared to Mannaway.

TABLE 15
20/40° C. Powder

| | 20° C. | | 40° C. | |
|---|---|---|---|---|
| | Comp. | Comp. | Comp. | Comp. |
| Stain | 3 | 4 | 3 | 4 |
| Carte Dór Chocolate Ice Cream | 1.4 | 2.8 | 2.1 | 0.5 |
| (Equest) | | | | |
| Vienetta (Equest) | 0.5 | 0.8 | 0.5 | n.d. |
| Chocolate Icecream | 0.9 | 0.9 | 1.1 | n.d. |
| L [CO] (Equest) | | | | |
| Porridge (EMPA 163 [CO]) | n.d. | n.d. | 1.3 | 5.1 |
| Cocoa (CFT CS02 [CO]) | 1.8 | 3.1 | n.d. | n.d. |
| Mayonnaise/Carbon black color | n.d. | 1.0 | n.d. | 2.7 |
| (CFT CS05S [CO]) | | | | |
| Salad dressing, with natural black | 2.0 | 4.8 | 1.3 | 5.1 |
| (CFT CS06 [CO]) | | | | |
| Sebum BEY with carbon black | 0.7 | 1.4 | 0.5 | 0.7 |
| (CFT CS32 [CO]) | | | | |
| Chocolate drink, pure | n.d. | 1.4 | n.d. | 0.8 |
| (CFT CS44 [CO]) | | | | |

Without limiting the scope and interpretation of the patent claims, certain technical effects of one or more of the aspects or embodiments disclosed herein are listed in the following: A technical effect is degradation or modification of mannan. Another technical effect is provision of mannanase which has good storage stability.

The foregoing description has provided by way of non-limiting examples of particular implementations and embodiments of the present disclosure a full and informative description of the best mode presently contemplated by the inventors for carrying out the invention. It is however clear to a person skilled in the art that the present disclosure is not restricted to details of the embodiments presented above, but that it can be implemented in other embodiments using equivalent means without deviating from the characteristics of the present disclosure.

Furthermore, some of the features of the above-disclosed aspects and embodiments of the present disclosure may be used to advantage without the corresponding use of other features. As such, the foregoing description should be considered as merely illustrative of the principles of the present disclosure, and not in limitation thereof. Hence, the scope of the present disclosure is only restricted by the appended patent claims.

In an embodiment at least one component of the compositions of the present disclosure has a different chemical, structural or physical characteristic compared to the corresponding natural component from which the at least one component is derived from. In an embodiment said characteristic is at least one of uniform size, homogeneous dispersion, different isoform, different codon degeneracy, different post-translational modification, different methylation, different tertiary or quaternary structure, different enzyme activity, different affinity, different binding activity, and different immunogenicity.

1. An enzyme composition comprising at least one mannanase enzyme having an amino acid sequence which has at least 70% sequence identity with SEQ ID NO: 16 (Man7), at least 93% sequence identity with SEQ ID NO: 12 (Man6), and/or at least 79% sequence identity with SEQ ID NO: 20 (Man14).

2. The enzyme composition of claim 1 further comprising:
a. at least one preservative selected from benzoic acid, sodium benzoate, hydroxybenzoate, citric acid, ascorbic acid, or a combination thereof;
b. optionally at least one polyol selected from propylene glycol, glycerol, a sugar, sugar alcohol, lactic acid, boric acid, boric acid derivative, aromatic borate ester, phenyl boronic acid derivative, peptide, or a combination thereof;
c. optionally at least one enzyme selected from proteases, amylases, cellulases, lipases, xylanases, mannanases, cutinases, esterases, phytases, DNAses, pectinases, pectinolytic enzymes, pectate lyases, carbohydrases, arabinases, galactanases, xanthanases, xyloglucanases, laccases, peroxidases and oxidases with or without a mediator, or a combination thereof; and
d. optionally at least one filler selected from maltodextrin, flour, sodium chloride, sulfate, sodium sulfate, or a combination thereof.

3. The enzyme composition of claim 1 in the form of a liquid composition or a solid composition such as solution, dispersion, paste, powder, granule, granulate, coated granulate, tablet, cake, crystal, crystal slurry, gel, or pellet.

4. A recombinant host cell comprising genetic elements that allow producing at least one recombinant polypeptide having mannanase activity and
at least 70% sequence identity with the amino acid sequence of SEQ ID NO: 16,
at least 93% sequence identity with the amino acid sequence of SEQ ID NO: 12, and/or
at least 79% sequence identity with the amino acid sequence of SEQ ID NO: 20,
and wherein the host cell is selected from the group consisting of:
fungal cells,
filamentous fungal cells from Division Ascomycota, Subdivision Pezizomycotina; preferably from the group consisting of members of the Class Sordariomycetes, Subclass Hypocreomycetidae, Orders Hypocreales and Microascales and Aspergillus, Chrysosporium, Myceliophthora and Humicola;
more preferably from the group consisting of Families Hypocreacea, Nectriaceae, Clavicipitaceae, Microascaceae, and Genera Trichoderma (anamorph of Hypocrea), Fusarium, Gibberella, Nectria, Stachybotrys, Claviceps, Metarhizium, Villosiclava, Ophiocordyceps, Cephalosporium, and Scedosporium;
more preferably from the group consisting of Trichoderma reesei (Hypocrea jecorina), T. citrinoviridae, T. longibrachiatum, T. virens, T. harzianum, T. asperellum, T. atroviridae, T. parareesei, Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Acremonium (Cephalosporium) chrysogenum, and Scedosporium apiospermum, and Aspergillus niger, Aspergillus awamori, Aspergillus oryzae, Chrysosporium lucknowense, Myceliophthora thermophila, Humicola insolens, and Humicola grisea,
bacterial cells, preferably gram positive Bacilli such as B. subtilis, B. licheniformis, B. megaterium, B. amyloliquefaciens, B. pumilus, gram negative bacteria such as Escherichia coli, actinomycetales such as Streptomyces sp., and
yeasts, such as Saccharomyces cerevisiae, Pichia pastoris, Yarrowia lipolytica,
most preferably Trichoderma reesei or Bacillus.

5. The recombinant host cell of claim 4, wherein the recombinant polypeptide is a fusion protein which, in addition to having the amino acid sequence having mannanase activity, comprises at least one of:
an amino acid sequence providing a secretory signal sequence, such as Bacillus amyloliquefaciens xylanase signal peptide;
an amino acid sequence which facilitates purification, such as an affinity tag, His-tag;
an amino acid sequence which enhances production, such as an amino acid sequence which is a carrier, such as CBM;
an amino acid sequence having an enzyme activity; and
an amino acid sequence providing for the fusion protein with binding affinity, such as a carbohydrate binding moiety.

6. A recombinant polypeptide having mannanase activity and obtainable by using the host cell of claim 4.

7. A method for producing mannanase comprising:
a. cultivating a recombinant host cell of claim 4, wherein
i. the genetic elements comprise at least one control sequence which controls the production of the recombinant polypeptide in the recombinant host cell under conditions that allow production of the polypeptide;
ii. the genetic elements optionally comprise at least one sequence encoding a signal sequence for transporting the polypeptide outside the host cell; and
iii. cultivating is carried out in conditions allowing production of the polypeptide; and
b. recovering the polypeptide.

8. A method for degrading or modifying mannan containing material comprising treating said mannan containing material with an effective amount of the enzyme composition of claim 1.

9. The method of claim 8 wherein the mannan containing material is plant based material, textile, waste water, sewage, oil, or a combination thereof.

10. An animal feed comprising the enzyme composition of claim 1, and at least one protein source of plant origin or a mannan containing product or by-product, and
a. Optionally at least one enzyme selected from protease, amylase, phytase, xylanase, endoglucanase, beta-glucanase, or a combination thereof; and
b. Optionally at least one filler selected from maltodextrin, flour, salt, sodium chloride, sulfate, sodium sulfate, or a combination thereof.

11. A feed supplement comprising the enzyme composition of claim 1; and
a. Optionally at least one enzyme selected from protease, amylase, phytase, xylanase, endoglucanase, beta-glucanase, or a combination thereof; and
b. Optionally at least one filler selected from maltodextrin, flour, salt, sodium chloride, sulfate, sodium sulfate or a combination thereof.

12. Use of the animal feed of claim 10 in:
a. feeding animals, preferably monogastric animals or ruminants; and/or
b. improving weight gain of animals.

13. A use of the enzyme composition of claim 1 in a detergent.

14. The use of claim 13 wherein the detergent is a liquid detergent or a dry detergent preferably in a form of a powder, bar, tablet, pouch, paste, gel, liquid, granule or granulate.

15. A use of the enzyme composition of claim 1 in oil drilling.

16. A use of the enzyme composition of claim 1 in processing coffee extract, fruit juice, pineapple juice, or soya milk.

17. The enzyme composition of claim 2 in the form of a liquid composition or a solid composition such as solution, dispersion, paste, powder, granule, granulate, coated granulate, tablet, cake, crystal, crystal slurry, gel, or pellet.

18. A recombinant polypeptide having mannanase activity and obtainable by using the host cell of claim 5.

19. A method for producing mannanase comprising:
a. cultivating a recombinant host cell of claim 5, wherein
i. the genetic elements comprise at least one control sequence which controls the production of the recombinant polypeptide in the recombinant host cell under conditions that allow production of the polypeptide;
ii. the genetic elements optionally comprise at least one sequence encoding a signal sequence for transporting the polypeptide outside the host cell; and
iii. cultivating is carried out in conditions allowing production of the polypeptide; and
b. recovering the polypeptide.

20. A method for degrading or modifying mannan containing material comprising treating said mannan containing material with an effective amount of the enzyme composition of claim 2.


SEQUENCE LISTING (48 sequences)

SEQ ID NO: 1 - DNA, 39 bp, Artificial Sequence
  primer
caaccgcctctgcagcttatgcacaaaacggatttcacg

SEQ ID NO: 2 - DNA, 39 bp, Artificial Sequence
  primer
cggtatatctctgtcttaatcactcttaagcccattttc

SEQ ID NO: 3 - DNA, 37 bp, Artificial Sequence
  primer
caaccgcctctgcagcttctgatggtcatagccaaac

SEQ ID NO: 4 - DNA, 36 bp, Artificial Sequence
  primer
cggtatatctctgtcttattggattgttacatgatc

SEQ ID NO: 5 - DNA, 40 bp, Artificial Sequence
  primer
caaccgcctctgcagctgcaagcgggttttatgtaaacgg

SEQ ID NO: 6 - DNA, 39 bp, Artificial Sequence
  primer
cggtatatctctgtcttatttaatggtaacgttatcaac

SEQ ID NO: 7 - DNA, 17 bp, Artificial Sequence
  primer
agctgcagaggcggttg

SEQ ID NO: 8 - DNA, 21 bp, Artificial Sequence
  primer
gacagagatataccgacagtg

SEQ ID NO: 9 - DNA, 975 bp, Bacillus clausii
atgaagagggaggacatggatcaaatgaaaagaaagcggttacaattgtttggaacactagtggtattggttttgttcgtgtacggtagcggttcggcatatgcacaaaacggatttcacgtatccggtacagagttgttggacaaaaatggcgatccttatgttatgcgtggcgtcaaccatggacactcttggtttaagcaagatctggaggaagcaatccctgccatagcagaaacaggggcgaacacggtgagaatggtcttatccaatggacagcaatgggaaaaagatgatgcctctgagcttgcccgtgtgctggctgccacagaaacatatggattgacaactgtgctggaagtccatgacgctacaggaagtgacgatcctgctgatttagagaaagcagtcgattattggatcgaaatggctgatgttctcaaggggacagaagaccgagtaatcattaacgttgccaatgaatggtatgggtcgtggaggagcgacgtttgggcagaagcatacgcacaagcgatcccgcgcttgcgcagcgctggcctctcccatacattaatggttgatgcggcaggttggggccagtaccctgcctccatccacgagcggggagccgatgtgtttgcgtccgatccattaaaaaacacgatgttttcgatccatatgtacgaatatgcaggagctgatagggcgacaattgcctataacattgatcgtgtgcttgctgaaaatcttgctgtggtgatcggtgaatttggccataggcatcatgatggcgatgtcgatgaagatgcgattttggcctatacagcagagcggcaagtgggctggctggcctggtcatggtatggcaacagcgggggtgttgaatacttggatttagctgaaggcccatcaggcccattgacgagttggggcaaacgaattgtttatggtgaaaatgggcttaagagtgattaa

SEQ ID NO: 10 - DNA, 870 bp, Bacillus clausii
cagaacggcttccacgtctccggcacggagctcctggacaagaacggcgacccttacgtcatgcgcggcgtcaaccacggccacagctggttcaagcaggacctcgaggaagccatccctgctatcgctgagacgggcgctaacacggtccgcatggtcctgagcaacggccagcagtgggagaaggacgacgctagcgagctggctcgcgtcctcgctgctacggagacgtacggcctcaccacggtcctggaggtccacgacgctacgggctcggacgaccccgccgacctcgagaaggccgtcgactactggatcgagatggctgacgtcctgaagggcaccgaggaccgcgtcatcatcaacgtcgccaacgagtggtacggctcctggcgcagcgacgtctgggccgaggcctacgctcaggctatccctcgcctccgctcggccggcctctcccacacgctcatggtcgacgctgccggctggggccagtaccctgcttccatccacgagcgcggcgctgacgtctttgcttcggaccccctgaagaacaccatgttctccatccacatgtacgagtacgctggcgctgaccgcgctaccatcgcctacaacatcgaccgcgtcctggctgagaacctggctgtcgtcatcggcgagtttggccaccgccaccacgacggcgacgtcgacgaggacgctatcctggcttacaccgccgagcgccaggtcggctggctggcttggtcgtggtacggcaactcgggcggcgtcgagtacctggacctggctgagggcccttcgggccctctcacgagctggggcaagcgcatcgtctacggcgagaacggcctgaagagcgactaa

SEQ ID NO: 11 - protein, 324 aa, Bacillus clausii
MKREDMDQMKRKRLQLFGTLVVLVLFVYGSGSAYAQNGFHVSGTELLDKNGDPYVMRGVNHGHSWFKQDLEEAIPAIAETGANTVRMVLSNGQQWEKDDASELARVLAATETYGLTTVLEVHDATGSDDPADLEKAVDYWIEMADVLKGTEDRVIINVANEWYGSWRSDVWAEAYAQAIPRLRSAGLSHTLMVDAAGWGQYPASIHERGADVFASDPLKNTMFSIHMYEYAGADRATIAYNIDRVLAENLAVVIGEFGHRHHDGDVDEDAILAYTAERQVGWLAWSWYGNSGGVEYLDLAEGPSGPLTSWGKRIVYGENGLKSD

SEQ ID NO: 12 - protein, 289 aa, Bacillus clausii
QNGFHVSGTELLDKNGDPYVMRGVNHGHSWFKQDLEEAIPAIAETGANTVRMVLSNGQQWEKDDASELARVLAATETYGLTTVLEVHDATGSDDPADLEKAVDYWIEMADVLKGTEDRVIINVANEWYGSWRSDVWAEAYAQAIPRLRSAGLSHTLMVDAAGWGQYPASIHERGADVFASDPLKNTMFSIHMYEYAGADRATIAYNIDRVLAENLAVVIGEFGHRHHDGDVDEDAILAYTAERQVGWLAWSWYGNSGGVEYLDLAEGPSGPLTSWGKRIVYGENGLKSD

SEQ ID NO: 13 - DNA, 1473 bp, Bacillus hemicellulosilyticus
atgagaaatttcggtaagttaattgtcagttcttgtcttctattcagtttttttctttttgcctctgatggtcatagccaaacacattctggtttttatatcgaaggttcaaccctttatgacgccaacggagagccctttgtaatgagaggtatcaatcatggacatgcctggtataaacatgattctaacgtcgctataccagctattgctaatcaaggagcaaatacaattcgtattgttctgtcagatggtggtcaatgggcaaaagatgatataaacacattaaatcaagtgctcgatttagcagaggaacatgagatgattgctgttgttgaggttcacgatgcaacaggatctaattctatggctgacttaaatcgtgctgtcgattattggattgaaatgaaagacgctttaattggaaaagaagatcgcgtcataattaacattgccaatgaatggtatggagcatgggacggacaaggctgggcaaatggctataaggaggttattccacgtttacgaaatgctggcttcactcatacattaatggtagatgcagctggttggggacaataccctcaatcgattcatgattatggtcaagaggtatttaatgctgatcctttagcaaatacgatgttttccatccatatgtatgaatatgctggcggaaatgcttcaatggtacaatctaatatcgatggtgtcgtcgatcaagggttagctcttgtaataggagaatttgggcatatgcatacggacggagatgttgatgaagcaacgatattgagctactcgcaacaaagaggagtcggttggctagcttggtcttggaaaggcaatgggactcaatgggaatatctagatttatcttatgattggcaaggaacaaacttaacttcttggggaaataccattgtccacgggcctaatggattactcgaaacatccattccaagctcgattttccataccgctccaaacaatggagatccccctcctcataacggtaatgaaacgatcttatatgatttcgaacatggcactcaaggctggtcaggttcttcacttcttggaggaccttggacgacgaatgaatggagtacaaatggtaaccattcattaaaggccgatattttcttatcagctaactccaaacatgaattagcaaaagttgaaaatcgaaatttatcaggctactctactttacaagccactgtccgccatgcacattggggaaatgttggtaatttaacggcgagaatgtatgtaaaaacgggctcaaactatagctggtttaatggtgatcctatcccagtaaactcagcaaatggtacgactgtcactttgcctctttcatctattccaaacctaaatgacgtaaaagaaattggcgttgaatttattggagcttcaaatagcaatggacaaaccgccatttatttagatcatgtaacaatccaataa

SEQ ID NO: 14 - DNA, 1395 bp, Bacillus hemicellulosilyticus
cagacccactcgggcttctacatcgagggctcgacgctctacgacgctaacggcgagccttttgtcatgcgcggcatcaaccacggccacgcctggtacaagcacgactccaacgtcgctatccctgctatcgctaaccagggcgctaacaccatccgcatcgtcctcagcgacggtggccagtgggccaaggacgacatcaacacgctgaaccaggtcctcgacctggccgaggagcacgagatgatcgctgtcgtcgaggtccacgacgctaccggctccaacagcatggccgacctcaaccgcgccgtcgactactggatcgagatgaaggacgccctgatcggcaaggaagaccgcgtcatcatcaacatcgctaacgagtggtacggcgcttgggacggccagggctgggccaacggctacaaggaagtcatccctcgcctgcgcaacgctggcttcacccacaccctcatggtcgacgctgccggctggggccagtaccctcagagcatccacgactacggccaagaggtcttcaacgccgaccctctggccaacaccatgttctccatccacatgtacgagtacgctggcggcaacgcctccatggtccagagcaacatcgacggcgtcgtcgaccagggcctcgctctggtcatcggcgagttcggccacatgcacacggacggcgacgtcgacgaggctaccatcctgagctactcgcagcagcgcggcgtcggctggctggcctggtcgtggaagggcaacggcacccagtgggagtacctcgacctgagctacgactggcagggcaccaacctcacgtcgtggggcaacacgatcgtccacggccctaacggcctcctggagacgtccatcccttccagcatctttcacaccgctcctaacaacggcgaccctcctccccacaacggcaacgagacgatcctgtacgacttcgagcacggcacgcagggctggtcgggctcgtccctgctgggcggcccttggaccaccaacgagtggtcgaccaacggcaaccactccctcaaggccgacatcttcctgtccgccaacagcaagcacgagctcgccaaggtcgagaaccgcaacctcagcggctactcgacgctgcaggctaccgtccgccacgctcactggggcaacgtcggcaacctgacggctcgcatgtacgtcaagacgggcagcaactactcgtggttcaacggcgaccccatccctgtcaactcggctaacggcaccaccgtcaccctccctctgagctcgatccccaacctcaacgacgtcaaggagatcggcgtcgagttcatcggcgctagcaacagcaacggccagaccgccatctacctggaccacgtcacgatccagtaa

SEQ ID NO: 15 - protein, 490 aa, Bacillus hemicellulosilyticus
MRNFGKLIVSSCLLFSFFLFASDGHSQTHSGFYIEGSTLYDANGEPFVMRGINHGHAWYKHDSNVAIPAIANQGANTIRIVLSDGGQWAKDDINTLNQVLDLAEEHEMIAVVEVHDATGSNSMADLNRAVDYWIEMKDALIGKEDRVIINIANEWYGAWDGQGWANGYKEVIPRLRNAGFTHTLMVDAAGWGQYPQSIHDYGQEVFNADPLANTMFSIHMYEYAGGNASMVQSNIDGVVDQGLALVIGEFGHMHTDGDVDEATILSYSQQRGVGWLAWSWKGNGTQWEYLDLSYDWQGTNLTSWGNTIVHGPNGLLETSIPSSIFHTAPNNGDPPPHNGNETILYDFEHGTQGWSGSSLLGGPWTTNEWSTNGNHSLKADIFLSANSKHELAKVENRNLSGYSTLQATVRHAHWGNVGNLTARMYVKTGSNYSWFNGDPIPVNSANGTTVTLPLSSIPNLNDVKEIGVEFIGASNSNGQTAIYLDHVTIQ

SEQ ID NO: 16 - protein, 464 aa, Bacillus hemicellulosilyticus
QTHSGFYIEGSTLYDANGEPFVMRGINHGHAWYKHDSNVAIPAIANQGANTIRIVLSDGGQWAKDDINTLNQVLDLAEEHEMIAVVEVHDATGSNSMADLNRAVDYWIEMKDALIGKEDRVIINIANEWYGAWDGQGWANGYKEVIPRLRNAGFTHTLMVDAAGWGQYPQSIHDYGQEVFNADPLANTMFSIHMYEYAGGNASMVQSNIDGVVDQGLALVIGEFGHMHTDGDVDEATILSYSQQRGVGWLAWSWKGNGTQWEYLDLSYDWQGTNLTSWGNTIVHGPNGLLETSIPSSIFHTAPNNGDPPPHNGNETILYDFEHGTQGWSGSSLLGGPWTTNEWSTNGNHSLKADIFLSANSKHELAKVENRNLSGYSTLQATVRHAHWGNVGNLTARMYVKTGSNYSWFNGDPIPVNSANGTTVTLPLSSIPNLNDVKEIGVEFIGASNSNGQTAIYLDHVTIQ

SEQ ID NO: 17 - DNA, 1449 bp, Virgibacillus soli
atgttattctctacttcactgtttacttctacttcaaaagcgaatgcagcaagcgggttttatgtaaacggaaacacactctatgacgcaacaggtaccccttttgtgataagaggaatcaatcatgctcactcttggtttaaagacgacacagcaaccgcaatacctgccattgcagcaactggggcgaatactattagaatcgtattatcggatggcagccaatatagtcgggatgatattgatggcgtgaggaatctaatatcattggctgaggaaaataatctaattgctatgttagaggtccacgatgctactggaaaagatgatatcagctcattagatagtgcggcagattattggattagtataaaagaagcacttatcggcaaggaagacaaagtcctaataaacatcgcaaatgaatggtacggtacttgggatggggctagttgggcggatggctacaaacaagtgattcccaaattaagaaatgcaggacttaaccacacactaatagtagactctgctggctgggggcaatttccggagtccattcacaattacggaaaagaagtattcaatgctgaccccctacaaaatacaatgttctctattcatatgtatgaatatgctggtggggacgcttctactgtcaaagcaaatattgacggtgtattaaatcaaggtctagccgtaatcattggagaatttggacataggcatacagacggagatgtagatgaagcaacaattatgaattattcccaagagaaaaatgttggctggctcgcatggtcgtggaaaggtaatggcatggaatgggattatttagacttatcctatgattgggccggaaataacctaaccgactggggaaataccattgtaaatagtacaaacggcttaaaagctacatctgaaataagtccagtatttggagatggagatgacggtgtaggcgacggtggtcctggggattctaacggaactgaaactacgctttataacttcgaaaccgggacagaaggatggagcggcgaaaatatagaaactggaccttggtcagtgaatgagtgggcagcaaaaggtaaccactctttaaaagctgatgttaatttgggtgataactctgaacattatctatacctaactcaaaacctaaattttagcggaaagtcacaactcacagcgactgtaaagcatgctgattggggaaacttcggggatgaaataaatgcaaagttatatgtaaaaacagaatcagattggcaatggtttgatggaggaattgaaaagatcaattcttcaattggaactattataaccttagatttatcatcgctctcaaacccaagtgatattaaagaagttggtgttcagtttacgggttcttcaaatagttatggcctaacagctttatatgttgataacgttaccattaaataa

SEQ ID NO: 18 - DNA, 1401 bp, Virgibacillus soli
gcctcgggcttctacgtcaacggcaacactctctacgacgccacgggcaccccatttgtcatccgcggcatcaaccacgctcactcgtggttcaaggacgacactgccaccgctatccctgctatcgctgctacgggcgccaacacgatccgcatcgtcctcagcgacggctcgcagtactcccgcgacgacatcgacggcgtccgcaacctcatctccctggccgaggagaacaacctcatcgccatgctggaggtccacgacgctaccggcaaggacgacatcagctcgctggacagcgccgccgactactggatctcgatcaaggaagccctcatcggcaaggaagacaaggtcctgatcaacatcgccaacgagtggtacggcacctgggacggcgctagctgggctgacggctacaagcaggtcatccctaagctccgcaacgccggcctcaaccacacgctcatcgtcgactcggctggctggggccagttcccggagagcatccacaactacggcaaggaagtcttcaacgccgaccccctgcagaacacgatgttctcgatccacatgtacgagtacgccggcggcgacgcttccacggtcaaggccaacatcgacggcgtcctcaaccagggcctggctgtcatcatcggcgagtttggccaccgccacaccgacggcgacgtcgacgaggccaccatcatgaactacagccaggagaagaacgtcggctggctggcttggagctggaagggcaacggcatggagtgggactacctcgacctgagctacgactgggccggcaacaacctcaccgactggggcaacacgatcgtcaactcgaccaacggcctgaaggccacctcggagatcagccctgtctttggcgacggcgacgacggcgtcggcgacggtggccccggcgacagcaacggcaccgagacgacgctgtacaactttgagacgggcaccgagggctggagcggcgagaacatcgagacgggcccttggtcggtcaacgagtgggctgccaagggcaaccactccctcaaggccgacgtcaacctgggcgacaacagcgagcactacctctacctgacgcagaacctcaacttctccggcaagtcgcagctgacggctaccgtcaagcacgctgactggggcaacttcggcgacgagatcaacgccaagctctacgtcaagaccgagagcgactggcagtggttcgacggtggcatcgagaagatcaactccagcatcggcaccatcatcacgctcgacctgtcgtccctgtcgaacccgtccgacatcaaggaagtcggcgtccagttcactggctcgtctaactcttacggcctcactgctctttacgtcgacaacgtcactatcaagtag

SEQ ID NO: 19 - protein, 482 aa, Virgibacillus soli
MLFSTSLFTSTSKANAASGFYVNGNTLYDATGTPFVIRGINHAHSWFKDDTATAIPAIAATGANTIRIVLSDGSQYSRDDIDGVRNLISLAEENNLIAMLEVHDATGKDDISSLDSAADYWISIKEALIGKEDKVLINIANEWYGTWDGASWADGYKQVIPKLRNAGLNHTLIVDSAGWGQFPESIHNYGKEVFNADPLQNTMFSIHMYEYAGGDASTVKANIDGVLNQGLAVIIGEFGHRHTDGDVDEATIMNYSQEKNVGWLAWSWKGNGMEWDYLDLSYDWAGNNLTDWGNTIVNSTNGLKATSEISPVFGDGDDGVGDGGPGDSNGTETTLYNFETGTEGWSGENIETGPWSVNEWAAKGNHSLKADVNLGDNSEHYLYLTQNLNFSGKSQLTATVKHADWGNFGDEINAKLYVKTESDWQWFDGGIEKINSSIGTIITLDLSSLSNPSDIKEVGVQFTGSSNSYGLTALYVDNVTIK

SEQ ID NO: 20 - protein, 466 aa, Virgibacillus soli
ASGFYVNGNTLYDATGTPFVIRGINHAHSWFKDDTATAIPAIAATGANTIRIVLSDGSQYSRDDIDGVRNLISLAEENNLIAMLEVHDATGKDDISSLDSAADYWISIKEALIGKEDKVLINIANEWYGTWDGASWADGYKQVIPKLRNAGLNHTLIVDSAGWGQFPESIHNYGKEVFNADPLQNTMFSIHMYEYAGGDASTVKANIDGVLNQGLAVIIGEFGHRHTDGDVDEATIMNYSQEKNVGWLAWSWKGNGMEWDYLDLSYDWAGNNLTDWGNTIVNSTNGLKATSEISPVFGDGDDGVGDGGPGDSNGTETTLYNFETGTEGWSGENIETGPWSVNEWAAKGNHSLKADVNLGDNSEHYLYLTQNLNFSGKSQLTATVKHADWGNFGDEINAKLYVKTESDWQWFDGGIEKINSSIGTIITLDLSSLSNPSDIKEVGVQFTGSSNSYGLTALYVDNVTIK

SEQ ID NO: 21 - DNA, 60 bp, Artificial Sequence
  primer
agtcaatcgcgacaagcgccagacccactcgggcttctacatcgagggctcgacgctcta

SEQ ID NO: 22 - DNA, 46 bp, Artificial Sequence
  primer
cgcgccggatccttactggatcgtgacgtggtccaggtagatggcg

SEQ ID NO: 23 - DNA, 60 bp, Artificial Sequence
  primer
agtcaatcgcgacaagcgccagaacggcttccacgtctccggcacggagctcctggacaa

SEQ ID NO: 24 - DNA, 51 bp, Artificial Sequence
  primer
cgcgccggatccttagtcgctcttcaggccgttctcgccgtagacgatgcg

SEQ ID NO: 25 - DNA, 1846 bp, Bacillus pumilus
atgaaaaaatgggttcaacgggtggcttgttttatgctgctgatcactttatgggcgggttggttcactctgaccgtaaaggcctcctcctatgtgcaaacatctggtacacattttgtattgaacaaccacccattttactttgctggcacaaataattattatttccattacaaatcaaaaaagatggtagatgctgtttttgacgatatgaaggcaatggatttaaaggttattcgtatttggggatttcacgatggtacccctcaagaaaactcagtcttacaatctcgtccaggtgtttatgaagaatccggttttcaaaaactagactatgcgatttataaagcagggcaggaaggaatcaagctggtcataccgctcgtgaacaattgggatgactttggcgggatgaatcaatatgtgaagtggtttcaggcaggatcacatgatcacttttatacagattctcggattaaaacagcttacaaaaactatgtgcgctatgtattagagagaaccaatacgtactcaggtgttcaatataaagatgaccctgctattatgacatgggagctcgccaatgagccgcgcgctcagtcagacccttcgggagatatactagtaaactgggcagatgaaatgagtgcatggatcaaatcaattgactcgaatcatcttgttgctgtaggagacgaagggttctttcgcatgacaggtcatgatgattggttttacagtggaggagaaggtgttgattgggatcgtttgactgctctccctcatattgattatggaacctatcatttatacccggatcactggaatcagtctgctgcatggggagtgaaatggatcaaagatcatatcacccgaggaaacgcaatcggaaaacctgttgtattagaagagtttggctatcaaaatcaagcagcccgtcctgatgtatatgatagctggctgaagacaattgaacagctcggaggcgcaggtagccaattttggattttaacaagcattcaagacgatgattccctctacccggattatgatggttttcgagttttaaaggagagccgggaggcaggaattattcgtgaacacgccaaaagaatgaatgaaaagaactgatgaagaatgcctgtttataaggaacttcatttgcataaaaaaattggatatggtatagtttttatggaaatgctaacgattaccgagacaagagtggggaaacccgctcttttgtattgaacaggcaatttttgtctcgacattattcatccgttttctgctccccctgctcacaataaagcagggtttttatgcagaatgattgataagagcgtttatcgaaagcacaaggaggaagagaatgagcaaaaaagtagtggatatcgtaagcgacatggtgcagccaattttagatggcttacagcttgaactcgttgatgttgaatttgtcaaagagggtcaaaactggttccttcgcgtatttattgactctgataaaggcgtcgatatcgaggagtgtgccaaagtgagcgaagccttgagcgaaaagcttgatgaggcagatccaattagccaaaactactttcttgaagtgtcctctcctggagcggagcgcccattaaagaaaaaagctgattttgaaaaagcacttggaaaaaatgttttcatgaaaacatacgaaccaattgatggtgaaaaggcatttgaaggtgagcttacaagctttgatggtgagattgcaacagtgacagtgaagatcaagacaagaaagaaagagatcaatattccatacgaaaaaattgctaacgcaagattagcagtttcgttcaattaa

SEQ ID NO: 26 - protein, 376 aa, Bacillus pumilus
MKKWVQRVACFMLLITLWAGWFTLTVKASSYVQTSGTHFVLNNHPFYFAGTNNYYFHYKSKKMVDAVFDDMKAMDLKVIRIWGFHDGTPQENSVLQSRPGVYEESGFQKLDYAIYKAGQEGIKLVIPLVNNWDDFGGMNQYVKWFQAGSHDHFYTDSRIKTAYKNYVRYVLERTNTYSGVQYKDDPAIMTWELANEPRAQSDPSGDILVNWADEMSAWIKSIDSNHLVAVGDEGFFRMTGHDDWFYSGGEGVDWDRLTALPHIDYGTYHLYPDHWNQSAAWGVKWIKDHITRGNAIGKPVVLEEFGYQNQAARPDVYDSWLKTIEQLGGAGSQFWILTSIQDDDSLYPDYDGFRVLKESREAGIIREHAKRMNEKN

SEQ ID NO: 27 - DNA, 1083 bp, Bacillus amyloliquefaciens
atgctcaaaaagttcgcagtctgtctgtctatcattttattactcatctcagccgcccgtccgatatcggctcacaccgtttaccctgtcaatcccaatgcccagcagacgacaaaagacgtcatgaactggctggcgcatttgcccaaccgttcagaaaacagggtcatgtccggtgcattcggcgggtacagcgatgtcaccttttcaatgacggaggaaaaccgcttgaaaaacgcgacgggacagtctcccgccatctacggctgtgattatgggagagggtggctggaaacatcggatatcaccgattctatcgactacagctgcaacagcagcctcatttcgtactggaaaagcggcggcctccctcaggtcagcctgcatctcgcaaatccggcctttccatcaggacactataaaacggccatttcaaacagccagtataaaaatatcctgaacccttcaactgttgaaggacggcggcttgaggccttgctcagcaaaatcgccgacggccttactcagctgaaaaatcaaggcgtcaccgttctgttcaggccgctgcatgagatgaacggtgaatggttctggtgggggctgacaggctacaaccaaaaagacactgagagaatctcgctgtacaaagagctttacaagaagatataccgctatatgacagagacaagaggattggataatcttttgtgggtgtattcgcctgatgccaacagagacttcaaaacagacttctacccaggctcatcttatgtggatattaccggactggatgcttacttcaccgacccgtatgcgatatcaggctatgatgaaatgctgtctctgaaaaaaccgtttgcctttgccgagaccggtccgtccggtaatatcggaagctttgattacgctgtttttatcaatgcgatcaggcaaaagtatcccgagacaacctactttttgacatgggatgaacaattaagcccggcagccaatcaaggcgcgcaaagcctttatcaaaacagctggacgctgaacaagggcgaaatgtggaatggcggaaccttgacgccgatcgcggaataa

SEQ ID NO: 28 - protein, 360 aa, Bacillus amyloliquefaciens
MLKKFAVCLSIILLLISAARPISAHTVYPVNPNAQQTTKDVMNWLAHLPNRSENRVMSGAFGGYSDVTFSMTEENRLKNATGQSPAIYGCDYGRGWLETSDITDSIDYSCNSSLISYWKSGGLPQVSLHLANPAFPSGHYKTAISNSQYKNILNPSTVEGRRLEALLSKIADGLTQLKNQGVTVLFRPLHEMNGEWFWWGLTGYNQKDTERISLYKELYKKIYRYMTETRGLDNLLWVYSPDANRDFKTDFYPGSSYVDITGLDAYFTDPYAISGYDEMLSLKKPFAFAETGPSGNIGSFDYAVFINAIRQKYPETTYFLTWDEQLSPAANQGAQSLYQNSWTLNKGEMWNGGTLTPIAE

SEQ ID NO: 29 - DNA, 1494 bp, Amphibacillus xylanus
gtgaagttaactaaactaaaactattgagtagtgtattttttgttgtattaactgtgttaatgttgtttgtccctgggaatattgtgaatgtaaaagctgctaacggcttttatgtaagcgattccaatctgtatgatgcaaatggaaatcaatttgttatgcgtggggttaatcatgcccattcatggtataaggacacgtataccgaggcaattcctgcaattgcggctacaggagcgaatactatccgaattgtattatctgatggagggcaataccaaaaagatgatataaacatagtcagaaatttgattgaaaccgcagaagccaataatttagtcgctgtacttgaggttcatgatgctactgggtcggattcattatcggatttgaaccgggctgtagattattggattgaaattaaagatgcgttaattggtaaagaagatacggtgatcataaacattgtcaatgaatggtatggcacttgggatggtcgtctctgggcagatggttataaacaggcgataccgagattaagagatgctggattaacacatacgttgatgattgatgcagcaggttgggggcaatttcctagctcgatccatcaatatggtagagaagtatttaatgcagatcgtttagggaatacaatgttttcgattcatatgtatgaatatgctggcggtgatgatcaaatggttagagataatattaacggtgtgatcaatcaagacttagctctagtgattggtgaatttggtcattatcacacagatggcgatgttgatgaagatacgattttgagttacgcggagcagacaggtgttggttggttagcatggtcatggaaaggcaatggaactgagtgggagtatcttgatctatcaaatgattggggaggaaattatttaacatcttggggtgacaggattgtaaatggagcaaatggattaagagaaacgagtcaaattgcttctgttttttcaggaaacaatggcgggactcctggaaatggtgaggaagagactcctggtgatgtaagtcatttcgcaaacttcgagaatggtactgaaggttgggaagcaagcaatgtatctggtggaccttgggcaacaaatgaatggagtgctagtggttcatatgctttaaaagccgatgcgcaattagcatctggaagagaacactatttatatcgaatcggtccctttaatttatctgggtcaacattaaacgcaacggtaaggggtgctaattgggggaattatggatctggtatcgacgtgaagctatacgttaagtacggagatggctggacgtggagagatagtggtgtacagacaattagagcgggagaatctattgatctatcactagatttatcaaatgttgatcgctcaaacattagagaagttggtatccagtttattggtggaaatcattcatctggaaaaaccgctttttatgttgatcatgtttattcacattag

SEQ ID NO: 30 - protein, 497 aa, Amphibacillus xylanus
VKLTKLKLLSSVFFVVLTVLMLFVPGNIVNVKAANGFYVSDSNLYDANGNQFVMRGVNHAHSWYKDTYTEAIPAIAATGANTIRIVLSDGGQYQKDDINIVRNLIETAEANNLVAVLEVHDATGSDSLSDLNRAVDYWIEIKDALIGKEDTVIINIVNEWYGTWDGRLWADGYKQAIPRLRDAGLTHTLMIDAAGWGQFPSSIHQYGREVFNADRLGNTMFSIHMYEYAGGDDQMVRDNINGVINQDLALVIGEFGHYHTDGDVDEDTILSYAEQTGVGWLAWSWKGNGTEWEYLDLSNDWGGNYLTSWGDRIVNGANGLRETSQIASVFSGNNGGTPGNGEEETPGDVSHFANFENGTEGWEASNVSGGPWATNEWSASGSYALKADAQLASGREHYLYRIGPFNLSGSTLNATVRGANWGNYGSGIDVKLYVKYGDGWTWRDSGVQTIRAGESIDLSLDLSNVDRSNIREVGIQFIGGNHSSGKTAFYVDHVYSH

SEQ ID NO: 31 - DNA, 1767 bp, Paenibacillus polymyxa
atgaaaaaactactgtcttgtctcatttcgctgtcaatgcttgtgtatatcttaccgacaatgatagtgtccgctaacaatgatggcgtaacgaaccttgctcttgattcaacacctagtgcgcaaagtgatattatttctgatgctgtctacaaaatcacagctcagcattcaggaaaaagccttgaggttgaaggcggttctaaagatgacggcgcgaatgttcaacaatggacagataacgggaaagaacagcagaaatggagagttgtggacgtcggtggcggatattacaagctcatcagtcaatctagcggaaaagcactggatgtggcaggtggtaatacacatgatggtgccaatgtgcaacagtggacggacaacggaaatgctcagcaaaagtggaagatcatcgatgtaggaggaggctattataagttgatctcacaaagctctggaaaggcactcgacgtcgttggtggttatacgcacgacggggccaatgtgcagcaatgggcagacaatggatctgctcaacagcgctggcgtttcacacaaattgatacaaccacggatacgacgccgccaacagcaccaacgaatttacaatcatcatcgaaaacaagtacctctgtaacattgacttggaccacaagcattgataatgtaggtgtgacaggctatgtcatttataatggaacagatttggtcgggacttctacaactacatcttatattgttacaggattaacagcgaacacttcctataacttcactgtcaaagcgaaggatgccgctgggaatatttcagaaccatcaaatgtcttgaaagtcacaacgagttcagattcttctcaaaacacaggtttttatgtgaagggcacaacattatatgatggaaacggtaatccatttgtgatgagaggaatcaatcatgcatacacatggtataaagggcaagaatcagtagcaattcctgcgattgcgaaaacgggtgcaaacaccatccggattgtcttatctgacggacagcagtggacaaaagatgatttaagcgcgcttcaaaatttgattacactcagtgagcaaaacaaacttgtagtgattttagaggtgcacgacggtactggcaatgacaatgccgcagttttaaataaaattgctgattattggattgaaatgaagtcagctttaattgggaaggaaaatacagttattttaaacatcgcaaatgaatggtttggtacatgggatggaaacggctgggcgcagggctacaaatcagtcataccaaagctgcgaaatgcgggcatcaaaaacacgattatggtggatgcggctggatggggacaatatccaaaatcgatttttgattacggaacgcaagtgttcgatgcagatccgctcaagaatacgatgttttccattcatatgtatgaatacgcaggcggcaacgcagaaacagtgaaaagtaatatcgacaacgtcctgaataaaaatcttgcactcatcattggagaatttggaattaaacatacaaacggagatgttgatgaagcaacgatcatgtcatacgcacagcaaaaaggtgttgggtatcttggctggtcatggaaaggaaatggttcaggtcttgaatatttagatatgagtaacgattgggctggcagcagttatacagagcaaggacatgccattatcgaaggaccaaatggcattcgtgcaacatcaaaattatcaaccatttacagcaatgggaaacaataa

SEQ ID NO: 32 - protein, 588 aa, Paenibacillus polymyxa
MKKLLSCLISLSMLVYILPTMIVSANNDGVTNLALDSTPSAQSDIISDAVYKITAQHSGKSLEVEGGSKDDGANVQQWTDNGKEQQKWRVVDVGGGYYKLISQSSGKALDVAGGNTHDGANVQQWTDNGNAQQKWKIIDVGGGYYKLISQSSGKALDVVGGYTHDGANVQQWADNGSAQQRWRFTQIDTTTDTTPPTAPTNLQSSSKTSTSVTLTWTTSIDNVGVTGYVIYNGTDLVGTSTTTSYIVTGLTANTSYNFTVKAKDAAGNISEPSNVLKVTTSSDSSQNTGFYVKGTTLYDGNGNPFVMRGINHAYTWYKGQESVAIPAIAKTGANTIRIVLSDGQQWTKDDLSALQNLITLSEQNKLVVILEVHDGTGNDNAAVLNKIADYWIEMKSALIGKENTVILNIANEWFGTWDGNGWAQGYKSVIPKLRNAGIKNTIMVDAAGWGQYPKSIFDYGTQVFDADPLKNTMFSIHMYEYAGGNAETVKSNIDNVLNKNLALIIGEFGIKHTNGDVDEATIMSYAQQKGVGYLGWSWKGNGSGLEYLDMSNDWAGSSYTEQGHAIIEGPNGIRATSKLSTIYSNGKQ

SEQ ID NO: 33 - DNA, 1470 bp, Bacillus hemicellulosilyticus
atggatatattaagaaagtgtgtacttgtactattggccttactattgttgttacctacgacatcaacggcattttctgaaagcgcttctactaatgagagagtgctaaatttatctgatccgaatgcgacacgctatacgaaggaattgtttgcgtttcttcaagacgtgagtggtgagcaagtgttgttcgggcaacagcatgcaacagatgaagggttgactctgacaggtgaaggaaatcgaattggttcaactgagtcggaggtgaagaatgcagtaggtgattatccagctgtttttgggtgggatacgaacagcttggatggtcgtgaaaagccaggtacagatgtggaaagtcaagagcaacgaattttaaatacagcagaatcgatgaaagtggcacatgaattaggagggatcatcacattaagtatgcatccggataactttgttaccggtcattactatggcgatacggatggtaacgtcgttcaagaaatattgccaggtggctccaagcacaatgaatttaacgcttggctagataatattgctgccctagcacatgaattagttgatgataatggagagcctattccggttatcttccgtccattccatgagcaaacaggttcgtggttttggtggggtgcgagcacaacaactcctgagcaatacaaagcgatttttcgatatacagtcgaatacttaagagatgcaaagggtgttcataactttttatatggattctcccctggtgcgggtcctgctggcgatctagatcgatatttagaaacgtacccaggtgataattatgtcgatatcttaggtattgataattatgatagtaagtcaaatgcggggtcagacgcttggttatctggaatggtaaaagatttagcgatgatctcgaaattagcagaggaaagagggaaggtatcagcctttactgaatttggatacagcgctgaagggatgagtcaaacgggtgatgcgttagattggtatacacgtgtgttaaatgcgataaaagcagatgaagatgcgcgaaacatatcctacatgctaacgtgggctaactttgggtggcctaataatatatttgttccgtatcgtgatgtgaatggggatttaggtggagatcatgagttattacctgactttgtacagttttatgaagatgaatactcagcatttcgtgaagatataaatgaaagtgtttacaatcgtaatgagagttatattgttgcggatcatgagccatttatgtatgttgtttcccctacgacaggtacatatataacaggctcgtctgttgtcttacgagcgaaagtagttaacgatgaggatccgtccgttacgtatcaagtggcgggttctgaagaagtctatgagatgactttagatgaaaatgggtattactctgctgattatattcctactgctcctaagaatggagctctgtag

SEQ ID NO: 34 - protein, 489 aa, Bacillus hemicellulosilyticus
MDILRKCVLVLLALLLLLPTTSTAFSESASTNERVLNLSDPNATRYTKELFAFLQDVSGEQVLFGQQHATDEGLTLTGEGNRIGSTESEVKNAVGDYPAVFGWDTNSLDGREKPGTDVESQEQRILNTAESMKVAHELGGIITLSMHPDNFVTGHYYGDTDGNVVQEILPGGSKHNEFNAWLDNIAALAHELVDDNGEPIPVIFRPFHEQTGSWFWWGASTTTPEQYKAIFRYTVEYLRDAKGVHNFLYGFSPGAGPAGDLDRYLETYPGDNYVDILGIDNYDSKSNAGSDAWLSGMVKDLAMISKLAEERGKVSAFTEFGYSAEGMSQTGDALDWYTRVLNAIKADEDARNISYMLTWANFGWPNNIFVPYRDVNGDLGGDHELLPDFVQFYEDEYSAFREDINESVYNRNESYIVADHEPFMYVVSPTTGTYITGSSVVLRAKVVNDEDPSVTYQVAGSEEVYEMTLDENGYYSADYIPTAPKNGAL

SEQ ID NO: 35 - DNA, 1110 bp, Bacillus alcalophilus
atgagaagtatgaagcttttatttgctatgtttattttagttttttcctcttttacttttaacttagtagttgcgcaagctagtggacatggacaaatgcataaagtaccttgggcaccccaagctgaagcacctggaaaaacggctgagaatggagtctgggataaagttagaaataatcctggaaaagccaatcctccagcaggaaaagtcaatggtttttatatagatggaacaaccttatatgatgcaaatggtaagccatttgtgatgcgcggaattaaccacgctcattcctggtacaagcctcacatagaaaccgcgatggaggcaattgctgatactggagcaaactccattcgtgtagttctctcagatggacaacagtggaccaaagatgatgttgacgaagtagcaaaaattatatctttagcagaaaaacattctttagttgctgttcttgaggtacatgatgcactcggaacagatgatattgaaccattacttaaaacagtcgattactggattgagatcaaagatgctttaatcggaaaagaggacaaagtaattattaacatttctaatgaatggtttggttcttggagcagtgaaggttgggcagaaggatataaaaaagcaattcctttactaagagaggcgggtctaaaacatacacttatggttgacgcagctgggtggggacaatttcctagatctattcatgaaaaaggattagacgtttttaactcagacccattaaagaatacaatgttttccattcatatgtatgaatgggcagcgggtaatcctcaacaagtaaaagacaatattgacggtgttcttgaaaagaatttagctgtagtaattggtgagttcggtcatcatcactacggaagagatgttgctgttgatacgatcttaagtcattcagagaagtatgatgtaggttggcttgcctggtcttggcacggaaatagtggtggtgtagagtatcttgacttagcaacagatttttcagggacgcaactaactgaatggggagaaagaattgtgtacggtccgaatggtttaaaagaaacttctgaaatcgttagtgtatacaaaaaataa

SEQ ID NO: 36 - protein, 369 aa, Bacillus alcalophilus
MRSMKLLFAMFILVFSSFTFNLVVAQASGHGQMHKVPWAPQAEAPGKTAENGVWDKVRNNPGKANPPAGKVNGFYIDGTTLYDANGKPFVMRGINHAHSWYKPHIETAMEAIADTGANSIRVVLSDGQQWTKDDVDEVAKIISLAEKHSLVAVLEVHDALGTDDIEPLLKTVDYWIEIKDALIGKEDKVIINISNEWFGSWSSEGWAEGYKKAIPLLREAGLKHTLMVDAAGWGQFPRSIHEKGLDVFNSDPLKNTMFSIHMYEWAAGNPQQVKDNIDGVLEKNLAVVIGEFGHHHYGRDVAVDTILSHSEKYDVGWLAWSWHGNSGGVEYLDLATDFSGTQLTEWGERIVYGPNGLKETSEIVSVYKK

SEQ ID NO: 37 - DNA, 1482 bp, Bacillus sp.
atgaaaaaaaagttatcacagatttatcatttaattatttgcacacttataataagtgtgggaataatggggattacaacgtccccatcagaagcaagttcaggcttttatgttgatggcaatacgttatatgacgcaaacgggcaaccatttgtcatgaaaggcattaaccatggacatgcttggtataaagacaccgcttcaacagctattcctgccattgcagagcaaggcgcgaacacgatacgtattgttttatcagatggcggtcaatgggaaaaagacgacattgacaccgttcgtgaagttattgagcttgcggagcaaaataaaatggtggctgtcgttgaagttcatgatgccacgggccgtgattcacgcagtgatttagatcgggcagtcgattattggatagagatgaaagatgcacttatcggcaaagaggatactgtcattattaacattgcaaacgaatggtatggcagttgggatggcgccgcttgggctgatggctacattgatgtcattccgaagcttcgcgatgccggcttaacacacaccttaatggttgatgcagcaggatgggggcaatatccgcaatctattcatgattacggacaagatgtgtttaatgcagatccgttaaaaaatacgatattctccatccatatgtatgagtatgctggtggtgatgctaacactgttagatcaaatattgatagagtcatagatcaagaccttgctctcgtaataggtgagttcggtcatagacacactgatggcgatgttgatgaagatacaatccttagttattctgaagaaactggcacaggatggctcgcttggtcttggaaaggcaacagtgccgaatgggattatttagacctttcagaagattgggctggtaaccatttaactgattggggaaataggattgtccacggggcaaatggcttgcaggaaacctccaaaccatccaccgtatttacagatgataacggtggtgcccctgaaccgccaactactactaccttgtatgactttgaaggaagcacacaagggtggcatggaagcaacgtgatgggtggcccttggtccgtaacagaatggggtgcgtcaggcaactactctttaaagggcgatgtcaatttaagctcaaattcttcacatgaactgtatagtgaacaaagtcgtaatctacacggatactctcagctaaatgcaaccgttcgccatgccaattggggaaatcccggtaatggcatgaatgcaagactttacgtgaaaacgggctctgattatacatggtatagcggtccttttacacgtatcaatagctccaactcaggtacaacgttatcttttgatttaaacaacatcgaaaatagtcatcatgttagggaaataggtgtgcaattttcagctgcagataatagcagcggtcaaactgctctatacgttgataatgttactttaagataa

SEQ ID NO: 38 - protein, 493 aa, Bacillus sp.
MKKKLSQIYHLIICTLIISVGIMGITTSPSEASSGFYVDGNTLYDANGQPFVMKGINHGHAWYKDTASTAIPAIAEQGANTIRIVLSDGGQWEKDDIDTVREVIELAEQNKMVAVVEVHDATGRDSRSDLDRAVDYWIEMKDALIGKEDTVIINIANEWYGSWDGAAWADGYIDVIPKLRDAGLTHTLMVDAAGWGQYPQSIHDYGQDVFNADPLKNTIFSIHMYEYAGGDANTVRSNIDRVIDQDLALVIGEFGHRHTDGDVDEDTILSYSEETGTGWLAWSWKGNSAEWDYLDLSEDWAGNHLTDWGNRIVHGANGLQETSKPSTVFTDDNGGAPEPPTTTTLYDFEGSTQGWHGSNVMGGPWSVTEWGASGNYSLKGDVNLSSNSSHELYSEQSRNLHGYSQLNATVRHANWGNPGNGMNARLYVKTGSDYTWYSGPFTRINSSNSGTTLSFDLNNIENSHHVREIGVQFSAADNSSGQTALYVDNVTLR

SEQ ID NO: 39 - DNA, 1551 bp, Bacillus circulans
atggggtggtttttagtgattttacgcaagtggttgattgcttttgtcgcatttttactgatgttctcgtggactggacaacttacgaacaaagcacatgctgcaagcggattttatgtaagcggtaccaaattattggatgctacaggacaaccatttgtgatgcgaggagtcaatcatgcgcacacatggtataaagatcaactatccaccgcaataccagccattgctaaaacaggtgccaacacgatacgtattgtactggcgaatggacacaaatggacgcttgatgatgtaaacaccgtcaacaatattctcaccctctgtgaacaaaacaaactaattgccgttttggaagtacatgacgctacaggaagcgatagtctttccgatttagacaacgccgttaattactggattggtattaaaagcgcgttgatcggcaaggaagaccgtgtaatcattaatatagctaacgagtggtacggaacatgggatggagtcgcctgggctaatggttataagcaagccatacccaaactgcgtaatgctggtctaactcatacgctgattgttgactccgctggatggggacaatatccagattcggtcaaaaattatgggacagaagtactgaatgcagacccgttaaaaaacacagtattctctatccatatgtatgaatatgctgggggcaatgcaagtaccgtcaaatccaatattgacggtgtgctgaacaagaatcttgcactgattatcggcgaatttggtggacaacatacaaacggtgatgtggatgaagccaccattatgagttattcccaagagaagggagtcggctggttggcttggtcctggaagggaaatagcagtgatttggcttatctcgatatgacaaatgattgggctggtaactccctcacctcgttcggtaataccgtagtgaatggcagtaacggcattaaagcaacttctgtgttatccggcatttttggaggtgttacgccaacctcaagccctacttctacacctacatctacgccaacctcaactcctactcctacgccaagtccgaccccgagtccaggtaataacgggacgatcttatatgatttcgaaacaggaactcaaggctggtcgggaaacaatatttcgggaggcccatgggtcaccaatgaatggaaagcaacgggagcgcaaactctcaaagccgatgtctccttacaatccaattccacgcatagtctatatataacctctaatcaaaatctgtctggaaaaagcagtctgaaagcaacggttaagcatgcgaactggggcaatatcggcaacgggatttatgcaaaactatacgtaaagaccgggtccgggtggacatggtacgattccggagagaatctgattcagtcaaacgacggtaccattttgacactatccctcagcggcatttcgaatttgtcctcagtcaaagaaattggggtagaattccgcgcctcctcaaacagtagtggccaatcagctatttatgtagatagtgttagtctgcaataa

SEQ ID NO: 40 - protein, 516 aa, Bacillus circulans
MGWFLVILRKWLIAFVAFLLMFSWTGQLTNKAHAASGFYVSGTKLLDATGQPFVMRGVNHAHTWYKDQLSTAIPAIAKTGANTIRIVLANGHKWTLDDVNTVNNILTLCEQNKLIAVLEVHDATGSDSLSDLDNAVNYWIGIKSALIGKEDRVIINIANEWYGTWDGVAWANGYKQAIPKLRNAGLTHTLIVDSAGWGQYPDSVKNYGTEVLNADPLKNTVFSIHMYEYAGGNASTVKSNIDGVLNKNLALIIGEFGGQHTNGDVDEATIMSYSQEKGVGWLAWSWKGNSSDLAYLDMTNDWAGNSLTSFGNTVVNGSNGIKATSVLSGIFGGVTPTSSPTSTPTSTPTSTPTPTPSPTPSPGNNGTILYDFETGTQGWSGNNISGGPWVTNEWKATGAQTLKADVSLQSNSTHSLYITSNQNLSGKSSLKATVKHANWGNIGNGIYAKLYVKTGSGWTWYDSGENLIQSNDGTILTLSLSGISNLSSVKEIGVEFRASSNSSGQSAIYVDSVSLQ

SEQ ID NO: 41 - DNA, 984 bp, Paenibacillus sp.
atgagacaacttttagcaaaaggtattttagctgcactggtcatgatgttagcgatgtatggattggggaatctctcttctaaagcttcggctgcaacaggtttttatgtaagcggtaccactctatatgattctactggtaaaccttttgtaatgcgcggtgtcaatcattcgcatacctggttcaaaaatgatctaaatgcagccatccctgctattgccaaaacaggtgcaaatacagtacgtatcgttttatctaatggtgttcagtatactagagatgatgtaaactcagtcaaaaatattatttccctggttaaccaaaacaaaatgattgctgttcttgaggtgcatgatgctaccggtaaagacgattacgcttctcttgatgccgctgtaaactactggatcagcatcaaagatgccttgattggcaaggaagatcgagtcattgttaatattgccaatgaatggtacggtacatggaatggcagtgcttgggcagatggttataagcaggctattcccaaactaagaaatgcaggcatcaaaaacactttaatcgttgatgccgccggctggggacaatgtcctcaatcgatcgttgattacgggcaaagtgtatttgcagcagattcgcttaaaaatacaattttctctattcacatgtatgaatatgcaggcggtacagatgcgatcgtcaaaagcaatatggaaaatgtactgaacaaaggacttcctttgatcatcggtgaatttggcgggcagcatacaaacggcgatgtagatgaacatgcaattatgcgttatggtcagcaaaaaggtgtaggttggctggcatggtcgtggtatggcaacaatagtgaactcagttatctggatttggctacaggtcccgccggtagtctgacaagtatcggcaatacgattgtaaatgatccatatggtatcaaagctacctcgaaaaaagcgggtatcttctaa

SEQ ID NO: 42 - protein, 327 aa, Paenibacillus sp.
MRQLLAKGILAALVMMLAMYGLGNLSSKASAATGFYVSGTTLYDSTGKPFVMRGVNHSHTWFKNDLNAAIPAIAKTGANTVRIVLSNGVQYTRDDVNSVKNIISLVNQNKMIAVLEVHDATGKDDYASLDAAVNYWISIKDALIGKEDRVIVNIANEWYGTWNGSAWADGYKQAIPKLRNAGIKNTLIVDAAGWGQCPQSIVDYGQSVFAADSLKNTIFSIHMYEYAGGTDAIVKSNMENVLNKGLPLIIGEFGGQHTNGDVDEHAIMRYGQQKGVGWLAWSWYGNNSELSYLDLATGPAGSLTSIGNTIVNDPYGIKATSKKAGIF

SEQ ID NO: 43 - DNA, 981 bp, Bacillus circulans
atggccaagttgcaaaagggtacaatcttaacagtcattgcagcactgatgtttgtcattttggggagcgcggcgcccaaagccgcagcagctacaggtttttacgtgaatggaggcaaattgtacgattctacgggtaaaccattttacatgaggggtatcaatcatgggcactcctggtttaaaaatgatttgaacacggctatccctgcgatcgcaaaaacgggtgccaatacggtacgaattgttttatcaaacggtacacaatacaccaaggatgatctgaattccgtaaaaaacatcattaatgtcgtaaatgcaaacaagatgattgctgtgcttgaagtacacgatgccactgggaaagatgacttcaactcgttggatgcagcggtcaactactggataagcatcaaagaagcactgatcgggaaggaagatcgggttattgtaaacattgcaaacgagtggtacggaacatggaacggaagcgcgtgggctgacgggtacaaaaaagctattccgaaattaagagatgcgggtattaaaaataccttgattgtagatgcagcaggctggggtcagtaccctcaatcgatcgtcgattacggacaaagcgtattcgccgcggattcacagaaaaatacggcgttttccattcacatgtatgagtatgcaggcaaggatgcggccaccgtcaaatccaatatggaaaatgtgctgaataaggggctggccttaatcattggtgagttcggaggatatcacaccaatggagatgtcgatgaatatgcaatcatgaaatatggtctggaaaaaggggtaggatggcttgcatggtcttggtacggtaatagctctggattaaactatcttgatttggcaacaggacctaacggcagtttgacgagctatggtaatacggttgtcaatgatacttacggaattaaaaatacgtcccaaaaagcgggaatcttttaa

SEQ ID NO: 44 - protein, 326 aa, Bacillus circulans
MAKLQKGTILTVIAALMFVILGSAAPKAAAATGFYVNGGKLYDSTGKPFYMRGINHGHSWFKNDLNTAIPAIAKTGANTVRIVLSNGTQYTKDDLNSVKNIINVVNANKMIAVLEVHDATGKDDFNSLDAAVNYWISIKEALIGKEDRVIVNIANEWYGTWNGSAWADGYKKAIPKLRDAGIKNTLIVDAAGWGQYPQSIVDYGQSVFAADSQKNTAFSIHMYEYAGKDAATVKSNMENVLNKGLALIIGEFGGYHTNGDVDEYAIMKYGLEKGVGWLAWSWYGNSSGLNYLDLATGPNGSLTSYGNTVVNDTYGIKNTSQKAGIF

SEQ ID NO: 45 - DNA, 1110 bp, Bacillus nealsonii
atggttgtgaaaaaattatcaagttttattctaattttactgttagttacttctgctttgtttattactgattcaaaagcaagtgctgcttcgggattttatgtaagcggtaccactttatatgatgcaacgggtaaaccgtttactatgagaggtgtaaatcatgctcattcttggtttaaagaagattcagcagctgctattccagcaatagcagcaactggagcaaacacagtaagaattgttttatctgatggtggacaatacaccaaagatgatattaatactgttaaaagccttttgtcattggcagaaaaaataaacttgcattctggagtcatgacgcacagaaaagacgatgtggaatctttaaatcgtgcagtcgattattggatcagcttaaaagacacattgataggcaaagaagataaagtgataataaacattgcgaatgaatggtatggtacttgggatggtgcggcatgggcagctggttataaacaagctattccaaagttacggaatgcaggcttaaatcatactctaataattgattctgctggatggggacaatacccagcttccattcataattatggaaaagaggtatttaatgcggatccattgaaaaatacaatgttctccatacatatgtatgagtacgctggtggggatgcagcaactgttaagtcaaatattgatggtgtcttaaaccaaggattagctttaataataggagagtttggacaaaaacatacaaatggagatgtagatgaagcaaccatcatgagttattcacagcaaaaaaatatcggttggcttgcatggtcttggaaaggaaatagcacagattggagctatctggatttaagcaacgattggtctggtaacagtttaactgattggggtaatacggttgttaatggggcaaatgggttaaaagccacttcaaaactaagcggagtattcggtagctcagcaggaacaaataatatattgtatgattttgaaagcggtaatcaaaactggactggatcaaatatcgcgggtggaccttggaacgaattcaagcttgatatcattcaggacgagcctcagactccagcgtaa

SEQ ID NO: 46 - protein, 369 aa, Bacillus nealsonii
MVVKKLSSFILILLLVTSALFITDSKASAASGFYVSGTTLYDATGKPFTMRGVNHAHSWFKEDSAAAIPAIAATGANTVRIVLSDGGQYTKDDINTVKSLLSLAEKINLHSGVMTHRKDDVESLNRAVDYWISLKDTLIGKEDKVIINIANEWYGTWDGAAWAAGYKQAIPKLRNAGLNHTLIIDSAGWGQYPASIHNYGKEVFNADPLKNTMFSIHMYEYAGGDAATVKSNIDGVLNQGLALIIGEFGQKHTNGDVDEATIMSYSQQKNIGWLAWSWKGNSTDWSYLDLSNDWSGNSLTDWGNTVVNGANGLKATSKLSGVFGSSAGTNNILYDFESGNQNWTGSNIAGGPWNEFKLDIIQDEPQTPA

SEQ ID NO: 47 - DNA, 984 bp, Bacillus circulans
atgatgttgatatggatgcagggatggaagtctattctagtcgcgatcttggcgtgtgtgtcagtaggcggtgggcttcctagtccagaagcagccacaggattttatgtaaacggtaccaagctgtatgattcaacgggcaaggcctttgtgatgaggggtgtaaatcatccccacacctggtacaagaatgatctgaacgcggctattccggctatcgcgcaaacgggagccaataccgtacgagtcgtcttgtcgaacgggtcgcaatggaccaaggatgacctgaactccgtcaacagtatcatctcgctggtgtcgcagcatcaaatgatagccgttctggaggtgcatgatgcgacaggcaaagatgagtatgcttcccttgaagcggccgtcgactattggatcagcatcaaaggggcattgatcggaaaagaagaccgcgtcatcgtcaatattgctaatgaatggtatggaaattggaacagcagcggatgggccgatggttataagcaggccattcccaaattaagaaacgcgggcattaagaatacgttgatcgttgatgcagcgggatgggggcaatacccgcaatccatcgtggatgagggggccgcggtatttgcttccgatcaactgaagaatacggtattctccatccatatgtatgagtatgccggtaaggatgccgctacggtgaaaacgaatatggacgatgttttaaacaaaggattgcctttaatcattggggagttcggcggctatcatcaaggtgccgatgtcgatgagattgctattatgaagtacggacagcagaaggaagtgggctggctggcttggtcctggtacggaaacagcccggagctgaacgatttggatctggctgcagggccaagcggaaacctgaccggctggggaaacacggtggttcatggaaccgacgggattcagcaaacctccaagaaagcgggcatttattaa

SEQ ID NO: 48 - protein, 327 aa, Bacillus circulans
MMLIWMQGWKSILVAILACVSVGGGLPSPEAATGFYVNGTKLYDSTGKAFVMRGVNHPHTWYKNDLNAAIPAIAQTGANTVRVVLSNGSQWTKDDLNSVNSIISLVSQHQMIAVLEVHDATGKDEYASLEAAVDYWISIKGALIGKEDRVIVNIANEWYGNWNSSGWADGYKQAIPKLRNAGIKNTLIVDAAGWGQYPQSIVDEGAAVFASDQLKNTVFSIHMYEYAGKDAATVKTNMDDVLNKGLPLIIGEFGGYHQGADVDEIAIMKYGQQKEVGWLAWSWYGNSPELNDLDLAAGPSGNLTGWGNTVVHGTDGIQQTSKKAGIY